SEQUENCE LISTING (6 sequences)

SEQ ID NO: 1 - protein, 453 aa, unidentified
  source 1..453: note = Heavy chain
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, unidentified
  source 1..214: note = Light chain
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 219 aa, synthetic construct
  source 1..219: note = Human IL-12 p35
MGPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 4 - protein, 328 aa, synthetic construct
  source 1..328: note = Human IL-12 p40
MGHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 5 - DNA, 660 bp, synthetic construct
  source 1..660: mol_type = other DNA; note = Human IL-12 p35
atgggtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 6 - DNA, 989 bp, synthetic construct
  source 1..989: mol_type = other DNA; note = Human IL-12 p40
atgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttagac

(19) United States
(12) Patent Application Publication
(10) Pub. No.: US 2026/0070964 A1
(43) Pub. Date: Mar. 12, 2026

(54) IL-12 GENE THERAPY AND ANTI-VEGF COMBINATION FOR TREATING CANCER

(71) Applicant: IMUNON, INC., Lawrenceville, NJ (US)

(72) Inventors: Khursheed ANWER, Madison, AL (US); Majed Mohd MATAR, Madison, AL (US); Jennifer RICE, Lawrenceville, NJ (US)

(21) Appl. No.: 19/109,485

(22) PCT Filed: Sep. 6, 2023
(86) PCT No.: PCT/US2023/073563

§ 371 Date: Mar. 6, 2025

Related U.S. Application Data
(60) Provisional application No. 63/374,900, filed on Sep. 7, 2022.

(51) Int. Cl. C07K 16/22; A61K 9/00; A61K 31/282; A61K 31/337; A61K 31/502; A61K 31/65; A61K 31/7088; A61K 38/20; A61K 47/34; A61P 35/00

(52) U.S. Cl. CPC C07K 16/22; A61K 9/0019; A61K 31/282; A61K 31/337; A61K 31/502; A61K 31/65; A61K 31/7088; A61K 38/208; A61K 47/34; A61P 35/00; C07K 2317/56

(57) ABSTRACT

The present disclosure relates to a combination therapy comprising an anti-VEGF antibody, a nanoparticle formulated plasmid comprising an IL-12 coding nucleic acid, and, optionally, at least one adjunctive chemotherapeutic drug, and methods of treatment using such combination therapies and/or compositions.

### CROSS-REFERENCE TO RELATED APPLICATIONS

The present application claims the priority benefit of U.S. Provisional Application 63/374,900, filed Sep. 7, 2022, which is hereby incorporated by reference in its entirety.

### REFERENCE TO SEQUENCE LISTING SUBMITTED ELECTRONICALLY VIA EFS-WEB

The content of the electronically submitted sequence listing in .XML file (Name: 2437_081PC01_SequenceListing_ST26; Size: 9,811 bytes; and Date of Creation: Sep. 6, 2023), filed with the application, is incorporated herein by reference in its entirety.

### FIELD OF THE DISCLOSURE

The present disclosure relates to the fields of cancer therapy, gene therapy, and immunology.

### BACKGROUND

Ovarian cancer is the fifth most lethal type of cancer among women in the United States, causing an estimated 14,000 deaths annually. There are approximately 22,000 new cases of ovarian cancer every year and the majority, approximately 70% of cases, are diagnosed in advanced stages III and IV. Epithelial Ovarian Cancer (EOC) is characterized by dissemination of tumor in the peritoneal cavity with a high risk of recurrence (75%, stage III and IV) after seemingly successful surgery and chemotherapy.

IL-12 is one of the most active cytokines for stimulating an immune response against cancer. However, when administered as a recombinant protein, the pharmacokinetics of IL-12 requires that it be administered by frequent, large bolus injections, resulting in serious toxicities that limit its use. The GEN-1 is an IL-12 DNA plasmid vector formulated using a lipopolymeric delivery system. GEN-1 can be delivered locally (e.g., intraperitoneally), offering the potential for cytokines to be expressed specifically in the tumor micro-environment with the goal of achieving increased efficacy while minimizing potential systemic toxicity. GEN-1 has been studied in subjects with recurrent ovarian cancer as a single agent or in combination with standard chemotherapy.

While traditional chemotherapy regimens are designed to inhibit tumor growth by cytotoxic mechanisms, immunocytokine therapies are designed to elicit tumor killing by enhancing the immune system against cancer cells. GEN-1 reduces the toxicity issues associated with IL-12. Its nanoparticle profile allows for cell transfection followed by persistent, local secretion of IL-12 at therapeutic levels, while avoiding the toxicities associated with recombinant IL-12.

There remains a need for ovarian cancer treatments, including those diagnosed with EOC as well as recurrent ovarian cancer.

### DESCRIPTION OF FIGURES

FIG. 1 shows the difference in tumor weight (mg) compared to a control group following the administration of mGEN-1, various dosage levels of Bevicuzimab, and mGEN-1 in combination with Bevacizumab at various dosage levels.

FIG. 2 shows the different tumor burden levels post tumor implant of untreated, nude-Foxn1^{nu} mice, nude-Foxn1^{nu} mice treated with doxorubicin (Doxil)+Bevicuzimab, and nude-Foxn1^{nu} mice treated with Doxil+Bevicuzimab+mGEN-1, plotted via IVIS signal quantification.

FIG. 3 shows the different tumor burden levels post tumor implant of untreated, nude-Foxn1^{nu} mice, nude-Foxn1^{nu} mice treated with Doxil+Bevicuzimab, and nude-Foxn 1^{nu} mice treated with Doxil+Bevicuzimab+mGEN-1, via IVIS whole body images.

FIG. 4 shows an exemplary hIL-12 expression plasmid.

FIG. 5 shows the PEG-PEI-Cholesterol structure.

FIG. 6 shows the dosing schedule of the neoadjuvant chemotherapy (NACT)+Bevacizumab arm and NACT+BEVACIZUMAB+GEN-1 arm of the clinical protocol.

FIG. 7 shows an overview of the schedule of the NACT+Bevacizumab arm and NACT+BEVACIZUMAB+GEN-1 arm of the clinical protocol.

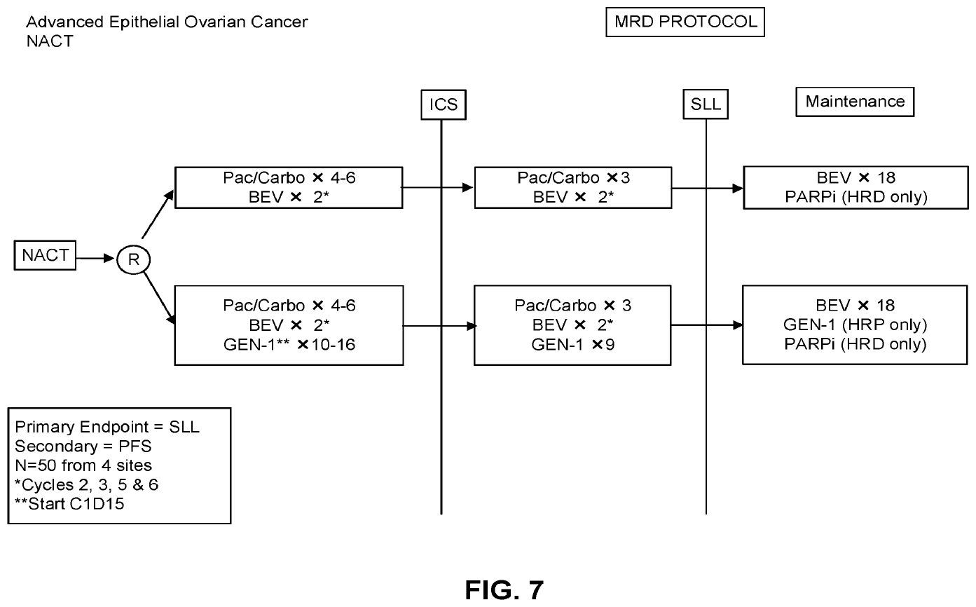

### BRIEF SUMMARY

Certain aspects of the disclosure are related to a combination therapy comprising: (i) a nucleic acid vector (e.g., a plasmid) comprising a polynucleotide that encodes an interleukin-12 (IL-12) formulated with a lipopolymer (e.g., a nanoparticle); and (ii) an antibody or antigen-binding fragment thereof that specifically binds a vascular endothelial growth factor (VEGF) (anti-VEGF antibody).

Certain aspects of the disclosure are related to a method of treating a subject suffering from cancer comprising administering to the subject a combination therapy comprising: (i) a nucleic acid vector (e.g., a plasmid) comprising a polynucleotide that encodes an interleukin-12 (IL-12) formulated with a lipopolymer (e.g., a nanoparticle); and (ii) an antibody or antigen-binding fragment thereof that specifically binds a vascular endothelial growth factor (VEGF) (anti-VEGF antibody).

In some aspects, the polynucleotide encodes human IL-12.

In some aspects, nucleic acid vector (e.g., a plasmid) comprises a promoter operably linked to a nucleic acid encoding a p35 subunit of IL-12 and a promoter operably linked to a nucleic acid encoding a p40 subunit of IL 12.

In some aspects, the promoter is a CMV promoter.

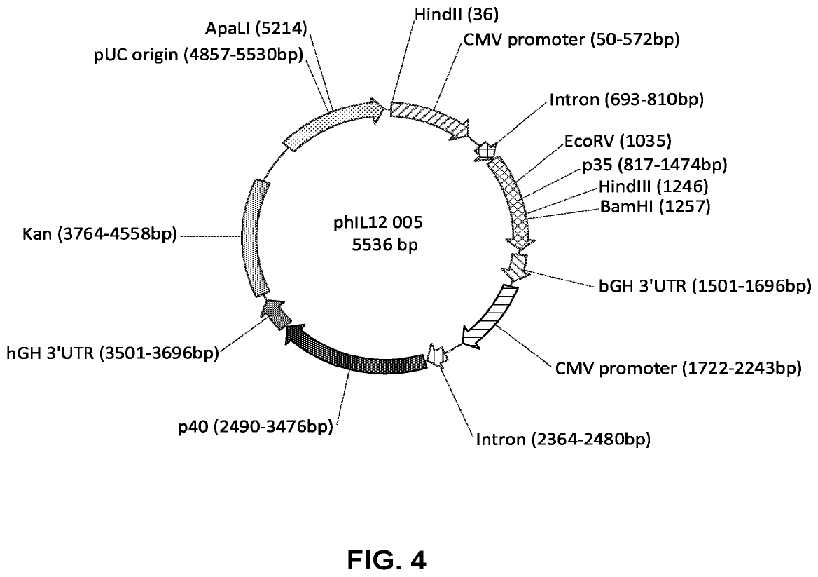

In some aspects, nucleic acid vector (e.g., a plasmid) comprises an intron, a 3′UTR (e.g., hGH 3′UTR), an antibiotic resistance gene, or any combination thereof (e.g., the elements of FIG. 4).

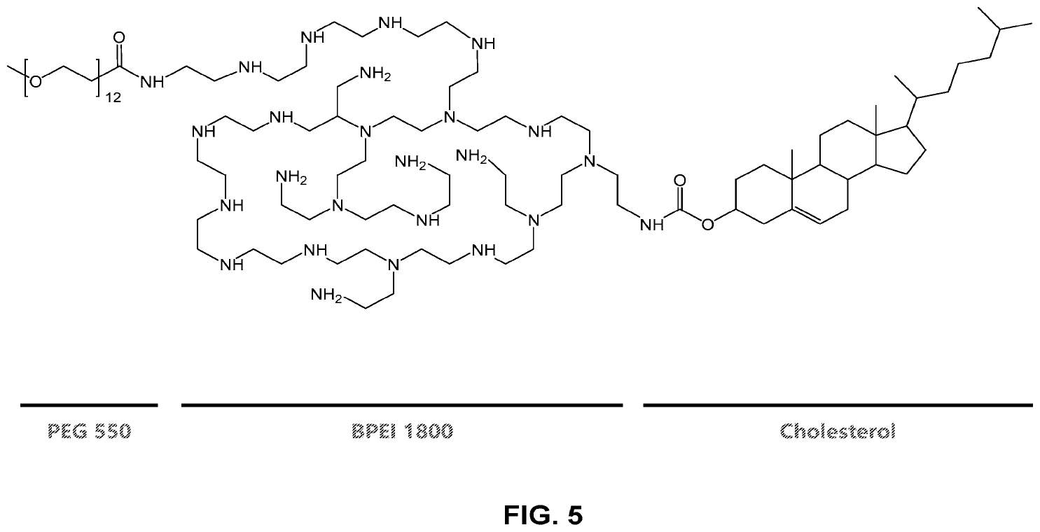

In some aspects, the lipopolymer comprises of polyethyleneimine (PEI) covalently linked independently to cholesterol and polyethylene glycol (PEG) groups (e.g., the lipopolymer of FIG. 5).

In some aspects, the combination further comprises an anticancer agent.

In some aspects, the anticancer agent is a chemotherapeutic agent.

In some aspects, the chemotherapeutic agent is selected from the group consisting of doxorubicin, paclitaxel, carboplatin, docetaxel, nab-paclitaxel, olaparib, and any combination thereof.

In some aspects, the anticancer agent is doxorubicin.

In some aspects, the anticancer agent is paclitaxel.

In some aspects, the anticancer agent is carboplatin.

In some aspects, the anticancer agent is docetaxel.

In some aspects, the anticancer agent is nab-paclitaxel.

In some aspects, the anticancer agent is olaparib.

In some aspects, the anti-VEGF antibody is selected from the group consisting of Bevacizumab (e.g., Avastin or a biosimilar thereof) or ranibizumab (e.g., Lucentis or a biosimilar thereof).

In some aspects, the anti-VEGF antibody comprises a variable heavy chain (VH) comprising an amino acid sequence with at least about 85% identity to SEQ ID NO: 1 (e.g., 90, 95, 96, 97, 98, 99, or 100% identity to SEQ ID NO: 1) and a variable light chain (VL) comprising an amino acid sequence with at least about 85% identity to SEQ ID NO: 2 (e.g., 90, 95, 96, 97, 98, 99, or 100% identity to SEQ ID NO: 2).

In some aspect, the method further comprises a surgery to remove all or part of a tissue or tumor (e.g., interval cytoreductive surgery) in the subject.

In some aspects, the nucleic acid vector formulated with the lipopolymer is administered intratumorally or intraperitoneally.

In some aspects, the nucleic acid vector formulated with the lipopolymer is administered intravenously.

In some aspects, the anti-VEGF antibody is administered intratumorally, intraperitoneally, intravenously, intravesicularly, or any combination thereof.

In some aspects, the anti-VEGF antibody is administered intratumorally or intraperitoneally.

In some aspects, the anti-VEGF antibody is administered intravenously.

In some aspects, the anti-VEGF antibody is administered intravesicularly.

In some aspects, the nucleic acid vector formulated with the lipopolymer is administered prior to, concurrently with, or after the anti-VEGF antibody.

In some aspects, the nucleic acid vector formulated with the lipopolymer is administered prior to, concurrently with, or after the anticancer agent.

In some aspects, an anticancer agent is administered (e.g., first), followed by the administration of the nucleic acid vector formulated with the lipopolymer (e.g., second), and followed by administration of the anti-VEGF antibody (e.g., third).

In some aspects, an anticancer agent is administered (e.g., first), followed by the administration of the nucleic acid vector formulated with the lipopolymer (e.g., second), followed by the anti-VEGF antibody (e.g. third), and followed by a surgery to remove all or part of a tissue or tumor (e.g., interval cytoreductive surgery) (e.g., fourth).

In some aspects, an anticancer agent is administered, followed by the administration of a DNA plasmid, followed by the anti-VEGF antibody, followed by an interval cytoreductive surgery.

In some aspects, the anticancer agent is administered prior to the interval cytoreductive surgery every three weeks for about 12 weeks to about 18 weeks.

In some aspects, the anticancer agent is administered at least about 28 days after the interval cytoreductive surgery (e.g., every three weeks for about 8-10 weeks, e.g., 9 weeks).

In some aspects, the administration of the anticancer agent comprises administering paclitaxel at a dose of about 100-200 mg/m² (e.g., about 175 mg/m²), optionally followed by administering carboplatin, e.g., at a dose of about AUC 5-6 IV.

In some aspects, the administration of the anticancer agent comprises administering docetaxel at a dose of 50-100 mg/m² (e.g., about 75 mg/m²), optionally followed by administering carboplatin, e.g., at a dose of about AUC 5-6 IV.

In some aspects, the administration of the anticancer agent comprises administering nab-paclitaxel at a dose of 200-300 mg/m² (e.g., about 260 mg/m²), optionally followed by administering carboplatin, e.g., at a dose of about AUC 5-6 IV.

In some aspects, the administration of the nanoparticle prior to the interval cytoreductive surgery begins 14-18 days (e.g., 15 days) after the first administration of the anticancer agent, e.g., every week for at least about 12 weeks to about 18 weeks.

In some aspects, the nanoparticle is administered at least about 28 days following the interval cytoreductive surgery, and administration begins 15 days after the first administration of the anticancer agent, every week for at least about 9 weeks.

In some aspects, the interleukin-12 (IL-12) formulated with a lipopolymer (e.g., a nanoparticle) is administered at a dose of about 35 mg/m² to about 80 mg/m².

In the aspects, the interleukin-12 (IL-12) plasmid formulated with a lipopolymer (e.g., a nanoparticle) is administered at a dose of about 80 mg/m².

In some aspects, the anti-VEGF antibody is administered prior to the interval cytoreductive surgery, at least about 22 days after the first administration of the anticancer agent, every week for at least about 12 weeks to up about 18 weeks.

In some aspects, the anti-VEGF antibody is administered at least about 28 days after the interval cytoreductive surgery, and at least about 22 days after the first administration of the anticancer agent, every week for at least about 9 weeks.

In some aspects, anti-VEGF antibody is administered at a dose of about 10-20 mg/kg IV (e.g., about 15 mg/kg IV).

In some aspects, the interval cytoreductive surgery (ICS) is administered at least about 28 days following the administration of an anticancer agent.

In some aspects, the interval cytoreductive surgery (ICS) is administered at least about 7 days following the administration of the DNA plasmid.

In some aspects, interval cytoreductive surgery (ICS) is administered at least about 7 days following the administration of the DNA plasmid.

In some aspects, the interval cytoreductive surgery (ICS) is administered at least about 28 days before the administration of the anti-VEGF antibody.

In some aspects, the interval cytoreductive surgery (ICS) is administered at least about 28 days following the administration of the anti-VEGF antibody.

In some aspects, the cancer is selected from a group consisting of ovarian cancer, fallopian tube cancer, primary peritoneal cancer, breast cancer, prostate cancer, colorectal cancer, bladder cancer, brain cancer (e.g., glioblastoma), lung cancer, and any combination thereof, and metastasis of any of the cancers.

In some aspects, the cancer is selected from a group consisting of ovarian cancer, fallopian tube cancer, primary peritoneal cancer, and any combination thereof.

In some aspects, the subject is a human.

### DETAILED DESCRIPTION

### I. Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Throughout this disclosure, various aspects are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “effective amount” or “pharmaceutically effective amount” or “therapeutically effective amount” as used herein refers to the amount or quantity of a drug or pharmaceutically active substance which is sufficient to elicit the required or desired therapeutic response, or in other words, the amount which is sufficient to elicit an appreciable biological response when administered to a patient.

“Transfecting” or “transfection” shall mean transport of nucleic acids from the environment external to a cell to the internal cellular environment, with particular reference to the cytoplasm and/or cell nucleus. Without being bound by any particular theory, it is to be understood that nucleic acids may be delivered to cells either after being encapsulated within or adhering to one or more cationic polymer/nucleic acid complexes or being entrained therewith. Particular transfecting instances deliver a nucleic acid to a cell nucleus. Nucleic acids include DNA and RNA as well as synthetic congeners thereof. Such nucleic acids include missense, antisense, nonsense, as well as protein producing nucleotides, on and off and rate regulatory nucleotides that control protein, peptide, and nucleic acid production. In particular, but not limited to, they can be genomic DNA, cDNA, mRNA, tRNA, rRNA, hybrid sequences or synthetic or semi-synthetic sequences, and of natural or artificial origin. In addition, the nucleic acid can be variable in size, ranging from oligonucleotides to chromosomes. These nucleic acids may be of human, animal, vegetable, bacterial, viral, or synthetic origin. They may be obtained by any technique known to a person skilled in the art.

As used herein, the term “pharmaceutical agent” or “drug” or any other similar term means any chemical or biological material or compound suitable for administration by the methods previously known in the art and/or by the methods taught in the present disclosure, which induce a desired biological or pharmacological effect, which may include but are not limited to (1) having a prophylactic effect on the organism and preventing an undesired biological effect such as preventing an infection, (2) alleviating a condition caused by a disease, for example, alleviating pain or inflammation caused as a result of disease, and/or (3) either alleviating, reducing, or completely eliminating a disease from the organism. The effect may be local, such as providing for a local anesthetic effect, or it may be systemic.

As used herein, the term “biocompatible” or “biodegradation” is defined as the conversion of materials into less complex intermediates or end products by solubilization hydrolysis, or by the action of biologically formed entities which can be enzymes and other products of the organism.

As used herein, “effective amount” means the amount of a nucleic acid or a bioactive agent that is sufficient to provide the desired local or systemic effect and performance at a reasonable risk/benefit ratio as would attend any medical treatment.

As used herein, “peptide” means peptides of any length and includes proteins. The terms “polypeptide” and “oligopeptide” are used herein without any particular intended size limitation, unless a particular size is otherwise stated.

As used herein, a “derivative” of a carbohydrate includes, for example, an acid form of a sugar, e.g. glucuronic acid; an amine of a sugar, e.g. galactosamine; a phosphate of a sugar, e.g. mannose-6-phosphate and the like.

As used herein, “administering” and similar terms mean delivering the composition to the individual being treated such that the composition is capable of being circulated systemically where the composition binds to a target cell and is taken up by endocytosis. Thus, the composition is preferably administered systemically to the individual, typically by subcutaneous, intramuscular, transdermal, intravenous, or intraperitoneal routes. Injectables for such use can be prepared in conventional forms, either as a liquid solution or suspension, or in a solid form that is suitable for preparation as a solution or suspension in a liquid prior to injection, or as an emulsion. Suitable excipients that can be used for administration include, for example, water, saline, dextrose, glycerol, ethanol, and the like; and if desired, minor amounts of auxiliary substances such as wetting or emulsifying agents, buffers, and the like.

As used herein, “efficacy” and similar terms means disappearance of tumor or shrinkage of tumor in size or reduction in tumor density or increase in lymphocyte count or increase in neutrophil count or improvement in survival, or all of the above.

As used herein, “toxicity” is defined as any treatment related adverse effects on clinical observation including but not limited to abnormal hematology or serum chemistry results or organ toxicity.

As used herein, the term “promoter/regulatory sequence” refers to a nucleic acid sequence required to express a gene product operably linked to a promoter/regulatory sequence. The term “constitutive” promoter refers to a nucleotide sequence that, when operably linked to a polynucleotide encoding or specifying a gene product, results in the production of a gene product in the cell under most or all physiological conditions of the cell. The term “inducible” promoter means that when operably linked to a polynucleotide encoding a specified gene product, it basically results in the production of a gene in the cell only when the inducer corresponding to the promoter is present in the cell the nucleotide sequence of the product.

As used herein, the term “expression” refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into messenger RNA (mRNA), and the translation of such mRNA into polypeptide(s). Expression of a gene produces a “gene product.”

As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

“As used herein, the term “expression vector” refers to a vector comprising a recombinant polynucleotide comprising an expression control sequence operably linked to the nucleotide sequence to be expressed. The expression vector contains sufficient cis-acting elements for expression; other elements for expression can be provided by the host cell or in an in vitro expression system. Expression vectors include expression vectors known in the art, including cosmids, plasmids (for example, naked or contained in liposomes), and viruses incorporating recombinant polynucleotides (for example, lentivirus, retrovirus, adenovirus, and adeno-associated virus).

As used herein, the term “operably linked” or “transcription control” refers to a functional linkage between a regulatory sequence and a heterologous nucleic acid sequence, which results in the expression of the latter. For example, when the first nucleic acid sequence and the second nucleic acid sequence are arranged in a functional relationship, the first nucleic acid sequence and the second nucleic acid sequence are operably linked. For example, if a promoter affects the transcription or expression of a coding sequence, the promoter is operably linked to the coding sequence. The operably linked DNA sequences may be adjacent to each other, and for example, in the case where two protein coding regions need to be linked, the DNA sequences are in the same reading frame.

As used herein, the term “transfer vector” refers to a composition containing an isolated nucleic acid and a substance that can be used to deliver the isolated nucleic acid to the inside of a cell. Many vectors are known in the art, including but not limited to linear polynucleotides, polynucleotides associated with ionic or amphiphilic compounds, plasmids, and viruses. The term transfer vector should also be interpreted to further include non-plasmid and non-viral compounds that facilitate the transfer of nucleic acids into cells, such as polylysine compounds, liposomes, and the like.

As used herein, the term “host cell” can be any type of cell, e.g., a primary cell, a cell in culture, or a cell from a cell line. In specific aspects, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

“Percent (%) amino acid sequence identity” with respect to a polypeptide sequence as set forth herein is defined as the percentage of amino acid residues in a candidate sequence of interest to be compared that are identical with the amino acid residues in a particular polypeptide sequence as set forth herein (e.g. a particular polypeptide sequence characterized by a sequence identifier in the sequence listings), after aligning the sequences and introducing gaps, if necessary, to achieve the maximum percent sequence identity, and not considering any conservative substitutions as part of the sequence identity. A sequence alignment performed for determining percent amino acid sequence identity can be carried out according to procedures known in the art, as described for example in EP 1 241 179 B1, which is incorporated herewith by reference, including in particular page 9, line 35 to page 10, line 40 with the definitions used therein and Table 1 regarding possible conservative substitutions. For example, a skilled person can use publicly available computer software. Computer program methods for determining sequence identity include, but are not limited to BLAST, BLAST-2, ALIGN or Megalign (DNASTAR) software. According to one embodiment, the software alignment program used can be BLAST. A skilled person can determine appropriate parameters for measuring alignment, including any algorithms needed to achieve maximal alignment over the full length of the sequences subjected to comparison. According to one embodiment, the % identity values can be generated using the WU-BLAST-2 computer program (Altschul et al., 1996, Methods in Enzymology 266:460-480, which is incorporated herewith by reference). According to one embodiment, the following parameters are used, when carrying out the WU-BLAST-2 computer program: Most of the WU-BLAST-2 search parameters are set to the default values. The adjustable parameters were set with the following values: overlap span=1, overlap fraction-0.125, word threshold (T)=11, and scoring matrix=BLOSUM62. The HSP S and HSP S2 parameters, which are dynamic values used by BLAST-2, are established by the program itself depending upon the composition of the sequence of interest and composition of the database against which the sequence is being searched. However, the values can be adjusted to increase sensitivity. A % sequence identity value can be determined by dividing (a) the number of matching identical amino acid residues between a particular amino acid sequence as set forth herein which is subjected to comparison (e.g. a particular polypeptide sequence characterized by a sequence identifier in the sequence listings) and the candidate amino acid sequence of interest to be compared, for example the number of matching identical amino acid residues as determined by WU-BLAST-2, by (b) the total number of amino acid residues of the polypeptide sequence as set forth herein which is subjected to comparison (e.g. a particular polypeptide sequence characterized by a SEQ. ID. NO. in the sequence listings).

“Percent (%) nucleic acid sequence identity” with respect to a nucleic acid sequence as set forth herein is defined as the percentage of nucleotides in a candidate sequence of interest to be compared that are identical with the nucleotides in a particular nucleic acid sequence as set forth herein (e.g. a particular polypeptide sequence characterized by a sequence identifier in the sequence listings), after aligning the sequences and introducing gaps, if necessary, to achieve the maximum percent sequence identity.

“As used herein, the term “homology” or “identity” refers to the identity of subunit sequence between two polymer molecules, for example, between two nucleic acid molecules, such as two DNA molecules or two RNA molecules, or between two polypeptide molecules. When subunit positions in two molecules are occupied by the same monomer subunit; for example, if the position of each of two DNA molecules is occupied by adenine, they are homologous or identical at that position. The homology between two sequences is a direct function of the number of matching or homologous positions; for example, if half of the positions in the two sequences (for example, 5 positions in a polymer of 10 subunits in length) are homologous, the two sequences are 50% homologous; if 90% of the positions (for example, 9 out of 10) are matched or homologous, then the two sequences are 90% homologous.

In the context of two or more nucleic acid or polypeptide sequences, identity percent refers to two or more sequences that are the same. When comparing and aligning for maximum correspondence in a comparison window or a designated area, as measured by using one of the following sequence comparison algorithms or by manual alignment and visual inspection, if the two sequences have a specified percentage of identical amino acid residues or nucleotides (e.g., 60% identity, optionally 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity over a specified region, or if not specified, over the entire sequence), then the two sequences are “substantially the same”. Optionally, the identity exists over a region of at least about 50 nucleotides (or 10 amino acids) in length, or more preferably over a region of 100 to 500 or 1000 or more nucleotides in length (Or 20, 50, 200 or more amino acids). For sequence comparison, usually a sequence serves as a reference sequence against which the test sequence is compared. When a sequence comparison algorithm is used, a test sequence and a reference sequence are input into a computer, and the sub-sequence coordinates and the sequence algorithm program parameters are specified, if necessary. Default program parameters can be used, or alternative parameters can be specified. Subsequently, the sequence comparison algorithm calculates the percent sequence identity of the test sequence relative to the reference sequence based on the program parameters. Methods of sequence alignment for comparison are well known in the art as disclosed above.

“Coding sequence” or a sequence “encoding” a particular molecule (e.g., a therapeutic molecule) is a nucleic acid that is transcribed (in the case of DNA) or translated (in the case of mRNA) into polypeptide, in vitro or in vivo, when operably linked to an appropriate regulatory sequence, such as a promoter. The boundaries of the coding sequence are determined by a start codon at the 5′ (amino) terminus and a translation stop codon at the 3′ (carboxy) terminus. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region.

A coding sequence can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the coding sequence.

As used herein, the term “recombinant DNA/RNA technology” refers to the manipulation of nucleic acid sequences outside of an organism. This technology comprises, but is not limited to, combining nucleic acid sequences (e.g., coding sequences, regulatory elements (e.g., promoters, enhancers, silencers, termination sequences), linkers (e.g., spacers, internal ribosome entry sites, cleavage sites) derived from a variety of sources, inserting nucleic acid sequences from a variety of sources in appropriate vectors (e.g., delivery vectors, expression vectors, integrating vectors), modifying or altering nucleotide sequences (e.g., by mutagenesis, insertion of modified nucleotides, 5′-capping, polyadenylation), synthesizing artificial nucleotide sequence. A variety of techniques well-known in the art (e.g., molecular cloning, polymerase chain reaction (PCR), digestion with restriction enzymes, in vitro ligation, mutagenesis, site-directed mutagenesis, prokaryotic and eukaryotic cell transformation or transduction, in vitro DNA/RNA synthesis, in vitro RNA-5′-capping, in vitro RNA-polyadenylation, complementary DNA (cDNA) synthesis, nucleic acid isolation, and the like) can be used to manipulate nucleic acid sequences outside an organism (see for example Green & Sambrook Molecular Cloning: A Laboratory Manual, volumes 1-3, 4th edition).

As used herein, the term “recombinant”, refers to any nucleic acid (e.g., DNA, or RNA), peptide (e.g., oligopeptide, polypeptide, or protein), cell, or organism, which is made by combining genetic material from two or more different sources. In some aspects, the recombinant nucleic acid, peptide, cell or organism comprises a portion of the genetic material from at least one source. In some aspects, “recombinant DNA” molecules can include DNA molecules derived from one organism and inserted in a host organism to produce new genetic combinations. In some aspects, “recombinant RNA” molecule (e.g., recombinant mRNA molecules) can include RNA molecules derived from one organism and inserted in a host organism to produce the expression of a desired genetic product in the host organism. In some aspects, “recombinant peptide” molecules can include amino acid molecules derived from an organism or cell, which are expressed from recombinant nucleic acid molecules.

As used herein, the term “isolated” means changed or removed from the natural state. For example, a nucleic acid or peptide naturally present in a living animal is not “isolated”, but the same nucleic acid or peptide that is partially or completely separated from a substance co-existing in its natural state is “isolated.” The isolated nucleic acid or protein may exist in a substantially purified form or may exist in a non-natural environment such as a host cell.

The term “tumor” as used herein refers to any mass of tissue that results from excessive cell growth or proliferation, either benign (non-cancerous) or malignant (cancerous), including pre-cancerous lesions.

The term “primary tumor”, as used herein, refers to the original, or first, tumor formed in the subject's body.

The term “metastasis”, “metastatic”, “secondary tumor”, or “metastatic tumor”, as used herein, refer to cancer (e.g., a tumor) formed by cancer cells derived from a primary cancer (e.g., tumor) that spread to further locations or areas of the body.

As used herein, the term “specifically binds” refers to an antigen binding molecule that recognizes and binds a protein of a binding partner (such as a tumor antigen) present in a sample, but the antigen binding molecule does not substantially recognize or bind to other molecules in the sample.

As used herein, the term “tumor heterogeneity” means that, after multiple divisions and proliferation during the growth of a tumor, daughter cells of the tumor its show molecular biological or genetic changes, so that there are differences in the growth rate, invasion ability, and drug sensitivity, prognosis and other aspects of the tumor. It is one of the characteristics of malignant tumors.

As used herein, the term “cancer” refers to a broad group of various diseases characterized by the uncontrolled growth of abnormal cells (e.g., malignant cells) in the body. Unregulated cell division and growth results in the formation of malignant tumors that invade neighboring tissues through local spread and can also metastasize to distant parts of the body through the lymphatic system or bloodstream. In some aspects, the methods of the present disclosure can be used to reduce the size of a primary tumor or a metastatic tumor, or treat a primary tumor or a metastatic tumor. The conditions that can be treated or prevented by the method of the present disclosure include, for example, various neoplasms, including benign or malignant tumors, various hyperplasias, and the like. The method of the present disclosure can achieve the inhibition and/or reversal of the undesirable hyperproliferative cell growth involved in such conditions. In some aspects, the cancer can be ovarian cancer.

As used herein, “ovarian cancer” refers to cancers arising in, or involving, the ovaries, e.g. in the epithelium of the ovaries. As used herein, the term “cancer” or “tumor” refers to an uncontrolled growth of cells which interferes with the normal functioning of the bodily organs and systems. A subject that has a cancer or a tumor is a subject having objectively measurable cancer cells present in the subject's body. Included in this definition are benign and malignant cancers, as well as dormant tumors or micrometastases. Cancers which migrate from their original location and seed vital organs can eventually lead to the death of the subject through the functional deterioration of the affected organs. Ovarian cancer is typically treated by cytoreductive surgery (also referred to herein as “debulking”) followed by administration of chemotherapy. As used here, “cytoreductive surgery” refers to surgical removal of at least part of the ovarian cancer tissue from a subject. Cytoreductive surgery can remove varying amounts of tumor tissue from a subject, depending upon the location and character of the tumor tissue, the health of the subject, and complicating factors which one of skill in the art can assess. In some embodiments, cytoreductive surgery can remove at least 10% of the tumor tissue, e.g. 10% or more, 20% or more, 30% or more, 40% or more, 50% or more, 60% or more, 70% or more, 80% or more, 90% or more, or 95% or more of the tumor tissue present in the subject.

As used herein, the term “transfected” or “transformed” or “transduced” refers to the process by which exogenous nucleic acid is transferred or introduced into a host cell. A “transfected” or “transformed” or “transduced” cell is a cell that has been transfected, transformed or transduced with exogenous nucleic acid. The cells include primary cell of a subject and progenies thereof.

As used herein, “refractory” as used herein refers to a disease, such as cancer, which does not respond to treatment. In an embodiment, a refractory cancer may be resistant to treatment before or at the beginning of the treatment. In other embodiments, a refractory cancer may become resistant during treatment. Refractory cancers are also called resistant cancers. In some aspects, refractory or recurrent malignant tumors can use the treatment methods disclosed herein.

As used herein, “relapsed” as used herein refers to the return of the signs and symptoms of a disease (e.g. cancer) or the return of a disease such as cancer during a period of improvement, for example, after a therapy, such as a previous treatment of cancer therapy.

As used herein, the term “combination therapy” means a therapy that includes at least a gene therapy, an anticancer agent, and an antibody with binding specificity for VEGF, which can be administered together or separately. In some aspects, compositions of the combination therapy are formulated together in a single composition or as separate compositions

As used herein, the terms “treat,” “treated,” and “treating” mean both therapeutic and prophylactic treatment or preventative measures wherein the object is to reverse, alleviate, ameliorate, lessen, inhibit, slow down progression, development, severity or recurrence of an undesired symptom, complication, condition, biochemical indicia of a disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. In some aspects, treatment includes eliciting a clinically significant response without excessive levels of side effects. In some aspects, treatment includes prolonging survival as compared to expected survival if not receiving treatment. As used herein, the term “amelioration” or “ameliorating” refers to a lessening of severity of at least one indicator of a condition or disease. As used herein, the term “preventing” or “prevention” refers to delaying or forestalling the onset, development or progression of a condition or disease for a period of time, including weeks, months, or years. As used herein, the term “prophylactic” (e.g., “prophylactic agent”, “prophylactic treatment”, “prophylactically effective amount”), refers to any complete or partial prevention of a disease or symptom thereof and/or can be therapeutic in terms of a partial or complete cure for a disease and/or adverse effect and/or symptom attributable to the disease.

As used herein, the terms “individual” and “subject” have the same meaning herein, and can be a human and animal from other species. As used herein, the terms “subject” and “patient” are used interchangeably. The subject can be an animal. In some aspects, the subject is a mammal such as a non-human animal (e.g., cow, pig, horse, cat, dog, rat, mouse, monkey or other primate, etc.). In some aspects, the subject is a human. In some aspects, the patient is a subject who has a disease, disorder, or condition, or is at risk of suffering from a disease, disorder, or condition, or is otherwise in need of the compositions and methods provided herein.

As used herein, the terms “therapeutically effective amount”, “therapeutically effective”, “effective amount” or “in an effective amount” are used interchangeably herein and refer to the amount of a compound, preparation, substance or composition that is effective to achieve a specific biological result as described herein, such as but not limited to treating or reducing the growth of a cancer or tumor. When indicating “immunologically effective amount”, “anti-tumor effective amount”, “tumor-suppressing effective amount” or “therapeutically effective amount”, the precise number of immune effector cells and therapeutic agents of the present disclosure to be administered can be determined by a physician in consideration of the individual's age, weight, tumor size, degree of infection or metastasis, and the condition of a patient (subject). An effective amount of immune effector cells refers to, but is not limited to, the number of immune effector cells which can increase, enhance or prolong the anti-tumor activity of immune effector cells; increase the number of anti-tumor immune effector cells or activated immune effector cells; promote tumor regression, tumor shrinkage and/or tumor necrosis.

The term “pharmaceutically acceptable” as used herein refers to those compounds, materials, compositions, formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “excipient” refers to any substance, not itself a therapeutic agent, which can be used in a composition for delivery of an active therapeutic agent to a subject or combined with an active therapeutic agent (e.g., to create a pharmaceutical composition) to improve its handling or storage properties or to permit or facilitate formation of a dose unit of the composition. Excipients include, but are not limited to, solvents, penetration enhancers, wetting agents, antioxidants, lubricants, emollients, substances added to improve appearance or texture of the composition and substances used to form hydrogels. Any such excipients can be used in any dosage forms according to the present disclosure. The foregoing classes of excipients are not meant to be exhaustive but merely illustrative as a person of ordinary skill in the art would recognize that additional types and combinations of excipients could be used to achieve the desired goals for delivery of a drug. The excipient can be an inert substance, an inactive substance, and/or a not medicinally active substance. The excipient can serve various purposes.

A person skilled in the art can select one or more excipients with respect to the particular desired properties by routine experimentation and without any undue burden. The amount of each excipient used can vary within ranges conventional in the art. Techniques and excipients which can be used to formulate dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21st edition, Gennaro, Ed., Lippincott Williams & Wilkins (2005).

As used herein, the term “immune response” refers to a biological response within an organism against a foreign agent or abnormal cell (e.g., a tumor cell), wherein the response protects the organism against such agents/cells and diseases caused by them. An immune response is mediated by the action of a cell of the immune system (e.g., a T lymphocyte (T cell), B lymphocyte (B cell), natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the organism's body of invading pathogens, cells or tissues infected with pathogens, cancerous or other abnormal cells, or, in cases of autoimmunity or pathological inflammation, normal human cells or tissues. In some aspects, an immune reaction includes, e.g., activation or inhibition of a T cell, e.g., an effector T cell or a Th cell, such as a CD4+ or CD8+ T cell, or the inhibition of a regulatory T cell (Treg cell).

As used herein, the term “autologous” refers to any material derived from an individual that will later be reintroduced into that same individual.

The term “antibody” herein is used in the broadest sense and encompasses various antibody structures, including but not limited to monoclonal antibodies, multispecific antibodies (e.g., bispecific antibodies), and antibody fragments so long as they exhibit the desired antigen-binding activity.

Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CH1). The term “Fab fragment” thus refers to an antibody fragment comprising a light chain comprising a VL domain and a CL domain, and a heavy chain fragment comprising a VH domain and a CH1 domain.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs), including the complementarity determining regions (CDRs) (see, e.g., Kindt et al. Kuby Immunology, 6th ed., W.H. Freeman and Co., page 91 (2007)).

A “paratope” or an “antigen binding site”, as used interchangeably herein, refers to a part of an antibody which recognizes and binds to an antigen. An antigen binding site is formed by several individual amino acid residues from the antibody's heavy and light chain variable domains arranged that are arranged in spatial proximity in the tertiary structure of the Fv region. In one embodiment, the antigen binding site is defined as a set of the six CDRs comprised in a cognate VH/VL pair.

The term “complementarity determining regions” or “CDRs” as used herein refers to each of the regions of an antibody variable domain which are hypervariable in sequence and contain antigen-contacting residues. Generally, antibodies comprise six CDRs: three in the VH domain (CDR-H1, CDR-H2, CDR-H3), and three in the VL domain (CDR-L1, CDR-L2, CDR-L3). Unless otherwise indicated, CDR residues and other residues in the variable domain (e.g., FR residues) are numbered herein according to the Kabat numbering system (Kabat et al., Sequences of Proteins of Immunological Interest, 5th Ed. Public Health Service, National Institutes of Health, Bethesda, Md., 1991).

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below.

“Framework” or “FR” as used herein refers to variable domain amino acid residues other than CDR residues. The framework of a variable domain generally consists of four framework domains: FRI, FR2, FR3, and FR4. Accordingly, the CDR and FR amino acid sequences generally appear in the following sequence in the (a) VH domain: FRI-CDR-H1-FR2-CDR-H2-FR3-CDR-H3-FR4; and (b) in the VL domain: FRI-CDR-L1-FR2-CDR-L2-FR3-CDR-L3-FR4.

Vascular endothelial growth factor (VEGF) is a homodimeric member of the cystine knot family of growth factors. Typically, VEGF refers to any native VEGF from any vertebrate source, including mammals such as primates (e.g. humans) and rodents (e.g., mice and rats), unless otherwise indicated. The term encompasses “full-length”, unprocessed VEGF as well as any form of VEGF that results from processing in the cell. The term also encompasses naturally occurring variants of VEGF, e.g., splice variants or allelic variants. A VEGF-dimer refers to a homodimer of two identical VEGF-molecules. A complex formed by two identical antibody molecules that are bound to a VEGF-dimer is herein referred to as VEGF-dimer-antibody-complex.

A “first and a second antigen binding site” comprised in a VEGF-dimer-antibody-complex refers to the antigen binding site that is comprised in the VH/VL pair of each one of the two antibodies comprised in the VEGF-dimer-antibody-complex. For example, while the antigen binding site of one of the two anti-VEGF antibodies in the VEGF-dimer-antibody-complex is the “first antigen binding site”, the antigen binding site of other one of the two anti-VEGF antibodies is automatically the “second antigen binding site”.

VEGF stimulates cellular responses by binding to tyrosine kinase receptors (the VEGF-receptors, or “VEGFRs”) on the cell surface, causing them to dimerize and become activated through transphosphorylation, although to different sites, times, and extents. VEGF-R1 and VEGF-R2 are closely related receptor tyrosine kinases (RTK). VEGF-A binds to VEGFR-1 (Flt-1), interacting with domain 2 of VEGF-R1, and VEGFR-2 (KDR/Flk-1), interacting with domains 2 and 3 of VEGF-R2.

The “VEGF-R1-binding region” and “VEGF-R2-binding region” of a VEGF molecule or a VEGF-dimer as used herein refers to those amino acids on the VEGF that interact with domain 2 of VEGF-RI or domains 2 or 3 of VEGF-R2, respectively.

As used herein, the terms “anti-VEGF antibody” and “an antibody that binds to VEGF” refer to an antibody or antigen-binding fragment thereof that is capable of binding VEGF with sufficient affinity such that the antibody is useful as a diagnostic and/or therapeutic agent in targeting VEGF. In one embodiment, the extent of binding of an anti-VEGF antibody to an unrelated, non-VEGF protein is less than about 10% of the binding of the antibody to VEGF as measured, e.g., by surface plasmon resonance (SPR). In certain embodiments, an antibody that binds to VEGF has a dissociation constant (KD) of <1 nM, or <0.15 nM. An antibody is said to “specifically bind” to VEGF when the antibody has a KD of 1 μM or less.

By “VEGFR-blocking selectivity” is used herein as an abbreviative term when referred to the property of anti-VEGF antibodies that preferentially inhibit binding to VEGF to VEGF-R2 rather than VEGF binding to VEGF-R1, when bound to a VEGF-dimer. Anti-VEGF antibodies that are capable of fully blocking VEGF-binding to VEGF-R2, but not fully block VEGF-binding to VEGF-R1, are considered to selectively block VEGF-signaling through VEGF-R2 but not through VEGF-R1, i.e. exhibit “VEGFR-blocking selectivity”.

As used herein, “affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein. Specific illustrative and exemplary embodiments for measuring binding affinity are described herein.

As used herein, the term “epitope” denotes the site on an antigen, either proteinaceous or non-proteinaceous, to which an anti-VEGF antibody binds. Epitopes can be formed both from contiguous amino acid stretches (linear epitope) or comprise non-contiguous amino acids (conformational epitope), e.g. coming in spatial proximity due to the folding of the antigen, i.e. by the tertiary folding of a proteinaceous antigen. Linear epitopes are typically still bound by an anti-VEGF antibody after exposure of the proteinaceous antigen to denaturing agents, whereas conformational epitopes are typically destroyed upon treatment with denaturing agents. An epitope comprises at least 3, at least 4, at least 5, at least 6, at least 7, or 8-10 amino acids in a unique spatial conformation.

Screening for antibodies binding to a particular epitope (i.e., those binding to the same epitope) can be done using methods routine in the art such as, e.g., without limitation, alanine scanning, peptide blots (see Meth. Mol. Biol. 248 (2004) 443-463), peptide cleavage analysis, epitope excision, epitope extraction, chemical modification of antigens (see Prot. Sci. 9 (2000) 487-496), and cross-blocking (see “Antibodies”, Harlow and Lane (Cold Spring Harbor Press, Cold Spring Harb., NY).

Antigen Structure-based Antibody Profiling (ASAP), also known as Modification-Assisted Profiling (MAP), allows to bin a multitude of monoclonal antibodies specifically binding to VEGF based on the binding profile of each of the antibodies from the multitude to chemically or enzymatically modified antigen surfaces (see, e.g., US 2004/0101920). The antibodies in each bin bind to the same epitope which may be a unique epitope either distinctly different from or partially overlapping with epitope represented by another bin. Also competitive binding can be used to easily determine whether an antibody binds to the same epitope of VEGF as, or competes for binding with, a reference anti-VEGF antibody. For example, an “antibody that binds to the same epitope” as a reference anti-VEGF antibody refers to an antibody that blocks binding of the reference anti-VEGF antibody to its antigen in a competition assay by 50% or more, and conversely, the reference antibody blocks binding of the antibody to its antigen in a competition assay by 50% or more. Also for example, to determine if an antibody binds to the same epitope as a reference anti-VEGF antibody, the reference antibody is allowed to bind to VEGF under saturating conditions. After removal of the excess of the reference anti-VEGF antibody, the ability of an anti-VEGF antibody in question to bind to VEGF is assessed. If the anti-VEGF antibody is able to bind to VEGF after saturation binding of the reference anti-VEGF antibody, it can be concluded that the anti-VEGF antibody in question binds to a different epitope than the reference anti-VEGF antibody. But, if the anti-VEGF antibody in question is not able to bind to VEGF after saturation binding of the reference anti-VEGF antibody, then the anti-VEGF antibody in question may bind to the same epitope as the epitope bound by the reference anti-VEGF antibody. To confirm whether the antibody in question binds to the same epitope or is just hampered from binding by steric reasons routine experimentation can be used (e.g., peptide mutation and binding analyses using ELISA, RIA, surface plasmon resonance, flow cytometry or any other quantitative or qualitative antibody-binding assay available in the art). This assay should be carried out in two set-ups, i.e. with both of the antibodies being the saturating antibody. If, in both set-ups, only the first (saturating) antibody is capable of binding to VEGF, then it can be concluded that the anti-VEGF antibody in question and the reference anti-VEGF antibody compete for binding to VEGF.

Sometimes two antibodies are deemed to bind to the same epitope if essentially all amino acid mutations in the antigen that reduce or eliminate binding of one antibody also reduce or eliminate binding of the other. Two antibodies are deemed to have “overlapping epitopes” if only a subset of the amino acid mutations that reduce or eliminate binding of one antibody reduce or eliminate binding of the other.

As used herein, the term “anti-tumor effect” refers to a biological effect that can be manifested in various ways, including but not limited to, for example, reduction in tumor volume, reduction in the number of tumor cells, reduction in the number of metastases, increase in life expectancy, reduction in tumor cell proliferation, and reduction in tumor cell survival rate, or improvement in various physiological symptoms related to cancerous conditions. The “anti-tumor effect” can also be expressed by the ability of the peptides, polynucleotides, cells and antibodies of the present disclosure to prevent or reduce the frequency of tumorigenesis.

The term “chemotherapy” or “chemotherapeutic agent” as used herein refers to a wide variety of chemotherapeutic agents that may be used in accordance with the present embodiments. The term “chemotherapy” refers to the use of drugs to treat cancer. A “chemotherapeutic agent” is used to connote a compound or composition that is administered in the treatment of cancer.

The term “pharmaceutical composition” refers to a preparation which is in such form as to permit the biological activity of the active ingredient to be effective, and which contains no additional components which are unacceptably toxic to a subject to which the composition would be administered. The composition can be sterile.

One skilled in the art will readily recognize that vectors, polynucleotides, and pharmaceutical compositions of the present disclosure, or combinations thereof, can be readily incorporated into one of the established kit formats which are well known in the art.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature.

### II. GEN-1 (Nanoparticle Formulated IL-12 DNA)

In animal models, recombinant IL-12 has been demonstrated to induce profound T-cell mediated antitumor effects causing regression of established tumors, followed by systemic immune memory. See The Oncologist, 1996, vol. 1, 88. However, systemic administration of recombinant IL-12 has resulted in dose limiting toxicity in several experimental trials and in an initial human trial. See Lab Invest., 1994, vol. 71, 862; Science, 1995, vol. 270, 908; J. Interferon Cytokine Res., 1995, vol. 14, 335. Dose limiting toxicity was also observed with intraperitoneal administration of recombinant IL-12 in a recent human clinical trial. Clin. Cancer Res., 2002, vol. 8, 3686. A gene delivery approach that can provide therapeutic levels of IL-12 locally at the tumor site would have the advantage of generating an anticancer response without causing systemic toxicity.

Both viral and non-viral gene delivery systems have been used for IL-12 gene delivery in animal models of cancer. The viral approach has serious practical limitations due to toxicity concerns mainly because of an increased incidence of cancer and a strong immune reaction to viral antigens by the host system. There is considerable interest in the development of non-viral gene delivery systems due to their lesser toxicity. Using polyvinylpyrrolidone (PVP), a non-viral gene delivery system, for the delivery of IL-12 to treat renal carcinoma (Renca) and colon cell carcinoma (CT26) has been demonstrated. See Gene Ther., 1999, vol. 6, 833. When tumors were subjected to this gene therapy, they displayed all the characteristics of IL-12 protein therapy, e.g., an increased infiltration of NK cells, CD4 and CD8 T cells, coupled with an increased expression of major histocompatibility complex (MHC) class I molecules. IL-12 gene delivery was well tolerated and highly effective against both Renca and CT26 tumor bearing animals. Tumor rejecting mice were also protected from a subsequent rechallenge, suggesting the presence of a long lasting systemic immunity. A functionalized and less toxic water soluble lipopolymer (WSLP) has been tested for delivery of the IL-12 gene to CT26 colon carcinoma tumors. See Mahato et al, Mol. Ther., 2001, vol. 4, 130. IL-12 plasmid (pIL-12) and WSLP (pIL-12/WSLP) treatment gave higher levels of intratumoral gene expression than naked DNA.

Interleukin-12 (IL-12) is a pro-inflammatory cytokine that plays an important role in innate and adaptive immunity. Gately, M K et al., Annu Rev Immunol. 16:495-521 (1998). IL-12 functions primarily as a 70 kDa heterodimeric protein consisting of two disulfide-linked p35 and p40 subunits. IL-12 p40 homodimers do exist, but other than functioning as an antagonist that binds the IL-12 receptor, they do not appear to mediate a biologic response. Id. The precursor form of the IL-12 p40 subunit (NM_002187; P29460; also referred to as IL-12B, natural killer cell stimulatory factor 2, cytotoxic lymphocyte maturation factor 2) is 328 amino acids in length, while its mature form is 306 amino acids long. The precursor form of the IL-12 p35 subunit (NM_000882; P29459; also referred to as IL-12A, natural killer cell stimulatory factor 1, cytotoxic lymphocyte maturation factor 1) is 219 amino acids in length and the mature form is 197 amino acids long. Id. The genes for the IL-12 p35 and p40 subunits reside on different chromosomes and are regulated independently of each other. Gately, M K et al., Annu Rev Immunol. 16:495-521 (1998). Many different immune cells {e.g., dendritic cells, macrophages, monocytes, neutrophils, and B cells) produce IL-12 upon antigenic stimuli. The active IL-12 heterodimer is formed following protein synthesis. Id.

Due to its ability to activate both NK cells and cytotoxic T cells, IL-12 protein has been studied as a promising anti-cancer therapeutic since 1994. See Nastala, C. L. et al., J Immunol 153:1697-1706 (1994). But despite high expectations, early clinical studies did not yield satisfactory results. Lasek W. et al., Cancer Immunol Immunother 63:419-435, 424 (2014). Repeated administration of IL-12, in most patients, led to adaptive response and a progressive decline of IL-12-induced interferon gamma (IFNy) levels in blood. Id. Moreover, while it was recognized that IL-12-induced anti-cancer activity is largely mediated by the secondary secretion of IFNy, the concomitant induction of IFNy along with other cytokines (e.g., TNF-a) or chemokines (IP-10 or MIG) by IL-12 caused severe toxicity. Id.

In addition to the negative feedback and toxicity, the marginal efficacy of the IL-12 therapy in clinical settings may be caused by the strong immunosuppressive environment in humans.

Furthermore, secondary effects of the cytokine IL-12 production, namely IFN-γ and nitric oxide (NO) levels were also higher in WSLP treated tumors when compared with naked DNA. A single injection of pIL-12/WSLP complexes produced suboptimal effects on tumor growth and animal survival, while repeated delivery yielded better efficacy which indicates insufficient delivery by the system. J. Control Release 2003, vol. 87, 177. Similarly, intratumoral injection of IL-12 plasmid in another polymeric carrier, PAGA, produced only partial inhibition of CT26 tumors. See Gene Ther., 2002, vol. 9, 1075. These results warrant the need for more efficient delivery systems. Despite their insufficiencies in earlier preclinical trials, the excellent molecular flexibility of polymeric gene carriers allows for complex modification and novel functionalization imperative for the development of more efficient gene delivery systems.

To achieve a desirable outcome from a combination approach involving gene therapeutics, the selection of an appropriate gene delivery system is important. The gene delivery system used in the aforementioned combination experiments (Molecular Therapy, 2004, vol. 9, 829) is a water soluble lipopolymer, PEI-Cholesterol (WSLP).

In some aspects, the DNA plasmid further encodes a synthetic polymer facilitating plasmid delivery that is a lipopolymer.

In some aspects, the lipopolymer further consists of polyethyleneimine (PEI) covalently linked independently to cholesterol and polyethylene glycol (PEG) groups.

The present disclosure provides a polymeric system, PEG-PEI-Cholesterol (PPC), which differs from WSLP (PEI-Cholesterol) in that it contains PEG moieties and yields significantly higher transfection efficiency in tumors. The addition of PEG is designed to enhance the stability of the nucleic acid/polymer complexes in the biological milieu to circumvent for this deficiency in the prior art (WSLP). Furthermore, the addition of PEG chains allows for the incorporation of ligands on to the PPC chain to improve the tissue selectivity of delivery. For example, the cholesterol moiety which is directly linked to the PEI back bone in the prior art (WSLP) may be extended farther from the PEI backbone to create a more flexible geometry for cell receptor interaction. Controlling the number of PEG molecules per unit of the PEI backbone is important to achieve optimal enhancement in transfection activity. A preferred range of composition was a PEG: PEI molar ratio of 2-4 at a fixed cholesterol content. The optimal ratio between PEI and cholesterol was 1:0.5 to 1:1.

Certain aspects of the disclosure are related to a combination therapy comprising: a (i) a nucleic acid vector (e.g., a plasmid) comprising a polynucleotide that encodes an interleukin-12 (IL-12) formulated with a lipopolymer (e.g., a nanoparticle); and (ii) an antibody or antigen-binding fragment thereof that specifically binds a vascular endothelial growth factor (VEGF) (anti-VEGF antibody).

Certain aspects of the disclosure are related to a method of treating a subject suffering from cancer comprising administering to the subject a combination therapy comprising: (i) a nucleic acid vector (e.g., a plasmid) comprising a polynucleotide that encodes an interleukin-12 (IL-12) formulated with a lipopolymer (e.g., a nanoparticle); and (ii) an antibody or antigen-binding fragment thereof that specifically binds a vascular endothelial growth factor (VEGF) (anti-VEGF antibody).

In some aspects, the polynucleotide encodes human IL-12. In some aspects, the polynucleotide encodes the p35 and p40 IL-12 subunits.

In some aspects, the human IL-12 p35 comprises an amino acid sequence having at least 70%, at least 75%, at least 80%, at least 85%, at least 90%, at least 95%, or 100% identity to SEQ ID NO: 3. In some aspects, the human IL-12 p40 comprises an amino acid sequence having at least 70%, at least 75%, at least 80%, at least 85%, at least 90%, at least 95%, or 100% identity to SEQ ID NO: 4.

In some aspects, the human IL-12 p35 comprises the sequence:

(SEQ ID NO: 3)
MGPARSLLLVATLVLLDHLSLARNLPVATPDPGMFPCLHHSQNLL
RAVSNMLQKARQTLEFYPCTSEEIDHEDITKDKTSTVEACLPLEL
TKNESCLNSRETSFITNGSCLASRKTSFMMALCLSSIYEDLKMYQ
VEFKTMNAKLLMDPKRQIFLDQNMLAVIDELMQALNFNSETVPQK
SSLEEPDFYKTKIKLCILLHAFRIRAVTIDRVMSYLNAS.

In some aspects, the human IL-12 p40 comprises the sequence:

(SEQ ID NO: 4)
MGHQQLVISWFSLVFLASPLVAIWELKKDVYVVELDWYPDAPGEM
VVLTCDTPEEDGITWTLDQSSEVLGSGKTLTIQVKEFGDAGQYTC
HKGGEVLSHSLLLLHKKEDGIWSTDILKDQKEPKNKTFLRCEAKN
YSGRFTCWWLTTISTDLTFSVKSSRGSSDPQGVTCGAATLSAERV
RGDNKEYEYSVECQEDSACPAAEESLPIEVMVDAVHKLKYENYTS
SFFIRDIIKPDPPKNLQLKPLKNSRQVEVSWEYPDTWSTPHSYFS
LTFCVQVQGKSKREKKDRVFTDKTSATVICRKNASISVRAQDRYY
SSSWSEWASVPCS.

In some aspects, the polynucleotide encoding the human IL-12 p35 has at least 70%, at least 75%, at least 80%, at least 85%, at least 90%, at least 95%, or 100% identity to SEQ ID NO: 5. In some aspects, the polynucleotide encoding the human IL-12 p35 has at least 70%, at least 75%, at least 80%, at least 85%, at least 90%, at least 95%, or 100% identity to SEQ ID NO: 6.

In some aspects, the polynucleotide encoding the human IL-12 p35 comprises the sequence:

(SEQ ID NO: 5)
atgggtccagcgcgcagcctcctccttgtggctaccctggtcctc
ctggaccacctcagtttggccagaaacctccccgtggccactcca
gacccaggaatgttcccatgccttcaccactcccaaaacctgctg
agggccgtcagcaacatgctccagaaggccagacaaactctagaa
ttttacccttgcacttctgaagagattgatcatgaagatatcaca
aaagataaaaccagcacagtggaggcctgtttaccattggaatta
accaagaatgagagttgcctaaattccagagagacctctttcata
actaatgggagttgcctggcctccagaaagacctcttttatgatg
gccctgtgccttagtagtatttatgaagacttgaagatgtaccag
gtggagttcaagaccatgaatgcaaagcttctgatggatcctaag
aggcagatctttctagatcaaaacatgctggcagttattgatgag
ctgatgcaggccctgaatttcaacagtgagactgtgccacaaaaa
tcctcccttgaagaaccggatttttataaaactaaaatcaagctc
tgcatacttcttcatgctttcagaattcgggcagtgactattgat
agagtgatgagctatctgaatgcttcctaa.

In some aspects, the polynucleotide encoding the human IL-12 p35 comprises the sequence:

(SEQ ID NO: 6)
Atgggtcaccagcagttggtcatctcttggttttccctggttttt
ctggcatctcccctcgtggccatatgggaactgaagaaagatgtt
tatgtcgtagaattggattggtatccggatgcccctggagaaatg
gtggtcctcacctgtgacacccctgaagaagatggtatcacctgg
accttggaccagagcagtgaggtcttaggctctggcaaaaccctg
accatccaagtcaaagagtttggagatgctggccagtacacctgt
cacaaaggaggcgaggttctaagccattcgctcctgctgcttcac
aaaaaggaagatggaatttggtccactgatattttaaaggaccag
aaagaacccaaaaataagacctttctaagatgcgaggccaagaat
tattctggacgtttcacctgctggtggctgacgacaatcagtact
gatttgacattcagtgtcaaaagcagcagaggctcttctgacccc
caaggggtgacgtgcggagctgctacactctctgcagagagagtc
agaggggacaacaaggagtatgagtactcagtggagtgccaggag
gacagtgcctgcccagctgctgaggagagtctgcccattgaggtc
atggtggatgccgttcacaagctcaagtatgaaaactacaccagc
agcttcttcatcagggacatcatcaaacctgacccacccaagaac
ttgcagctgaagccattaaagaattctcggcaggtggaggtcagc
tgggagtaccctgacacctggagtactccacattcctacttctcc
ctgacattctgcgttcaggtccagggcaagagcaagagagaaaag
aaagatagagtcttcacggacaagacctcagccacggtcatctgc
cgcaaaaatgccagcattagcgtgcgggcccaggaccgctactat
agctcatcttggagcgaatgggcatctgtgccctgcagttagac.

In some aspects, nucleic acid vector (e.g., a plasmid) comprises a promoter operably linked to a nucleic acid encoding a p35 subunit of IL-12 and a promoter operably linked to a nucleic acid encoding a p40 subunit of IL 12.

In some aspects, nucleic acid vector (e.g., a plasmid) comprises an intron, a 3′UTR (e.g., hGH 3′UTR), an antibiotic resistance gene, or any combination thereof (e.g., the elements of FIG. 4).

In some aspects, the lipopolymer comprises of polyethyleneimine (PEI) covalently linked independently to cholesterol and polyethylene glycol (PEG) groups (e.g., the lipopolymer of FIG. 5).

In some aspects, the nanoparticle disclosed herein comprises a DNA plasmid that encodes human IL-12.

In some aspects, wherein nanoparticle comprises a synthetic polymer facilitating plasmid delivery that is a lipopolymer.

In some aspects, the lipopolymer further comprises polyethyleneimine (PEI) covalently linked independently to cholesterol and polyethylene glycol (PEG) groups

In some aspects, the gene delivery polymer is a cationic polymer or a non-condensing polymer. The cationic polymer is selected from the group comprising polylysine, polyethylenimine, functionalized derivatives of polyethylenimine (PEI), polypropylenimine, aminoglycoside-polyamine, dideoxy-diamino-b-cyclodextrin, spermine and spermidine. One example of a cationic gene delivery polymer suitable for the present disclosure is a PEI derivative comprising a PEI backbone, a lipid, and a hydrophilic polymer spacer wherein the lipid is directly bound to the polyethylenimine backbone or covalently bound to the polyethylene glycol spacer, which in turn is bound, via a bio compatible bond, to the PEI.

The cationic gene delivery polymer of the present disclosure may further comprise a targeting moiety including antibodies or antibody fragments, cell receptors, growth factor receptors, cytokine receptors, folate, transferrin, epidermal growth factor (EGF), insulin, asialoorosomucoid, mannose-6-phosphate (monocytes), mannose (macrophage, some B cells), Lewis× and sialyl LewisX (endothelial cells), N-acetyllactosamine (T cells), galactose (colon carcinoma cells), and thrombomodulin (mouse lung endothelial cells), fusogenic agents such as polymixin B and hemaglutinin HA2, lysosomotrophic agents, nucleus localization signals (NLS) such as T-antigen, and the like. Another gene delivery polymer is a non-condensing polymer selected from the group comprising polyvinylpyrrolidone, polyvinylalcohol, poly(lactide-co-glycolide) (PLGA) and triblock copolymers of PLGA and PEG. The gene delivery polymer may also be a non-condensing polymer. Examples of such non-condensing polymers include polyvinyl pyrollidone, polyvinyl alcohol, poloxamers, polyglutamate, gelatin, polyphosphoesters, silk-elastin-like hydrogels, agarose hydrogels, lipid microtubules, poly(lactide-co-glycolide) and polyethyleneglycol-linked poly(lactide-co-glycolide).

The gene delivery polymer is a cationic polymer or a non-condensing polymer. The cationic polymer is selected from the group comprising polylysine, polyethylenimine, functionalized derivatives of polyethylenimine, polypropylenimine, aminoglycoside-polyamine, dideoxy-diamino-b-cyclodextrin, spermine and spermidine. One example of a cationic gene delivery polymer suitable for the present invention is a polyethylenimine derivative comprising a polyethylenimine (PEI) backbone, a lipid, and a polyethylene glycol spacer wherein the lipid is directly bound to the polyethylenimine backbone or covalently bound to the polyethylene glycol spacer, which in turn is bound, via a biocompatible bond, to the PEI.

In some aspects, the nanoparticle that comprises a DNA plasmid that encodes interleukin-12 (IL-12) and a synthetic polymer facilitating plasmid delivery, is delivered intraperitoneally.

In some aspects, the nanoparticle is administered at a dose of about 35 mg/m² to about 80 mg/m². In some aspects, the nanoparticle is administered at a dose of about 50 mg/m² to about 100 mg/m². In some aspects, the nanoparticle is administered at a dose of about 80 mg/m².

### III. Anti-VEGF Antibody

In another aspect of the foregoing combination therapy the pharmaceutical agent is an anti-VEGF antibody.

In some aspects, the anti-VEGF antibody is selected from the group consisting of Bevacizumab (e,g., Avastin or a biosimilar thereof) or ranibizumab (e.g., Lucentis or a biosimilar thereof).

In some aspects, the antibody with binding specificity for vascular endothelial growth factor (VEGF) is Bevacizumab or a biosimilar thereof.

In some aspects, the anti-VEGF antibody comprises a first amino acid sequence having a sequence with at least about 85% identity to SEQ ID NO: 1 (e.g., 90, 95, 96, 97, 98, 99 or 100% identity to SEQ ID NO:1) and a second amino acid sequence having a sequence with at least about 85% identity to SEQ ID NO:2 (e.g., 90, 95, 96, 97, 98, 99 or 100% identity to SEQ ID NO:2).

In some aspects the heavy chain of the anti-VEGF antibody comprises the sequence:

(SEQ ID NO: 1)
EVQLVESGGGLVQPGGSLRLSCAASGYTFTNYGMNWVRQAPGKGL
EWVGWINTYTGEPTYAADFKRRFTFSLDTSKSTAYLQMNSLRAED
TAVYYCAKYPHYYGSSHWYFDVWGQGTLVTVSSASTKGPSVFPLA
PSSKSTSGGTAALGCLVKDYFPEPVTVSWNSGALTSGVHTFPAVL
QSSGLYSLSSVVTVPSSSLGTQTYICNVNHKPSNTKVDKKVEPKS
CDKTHTCPPCPAPELLGGPSVFLFPPKPKDTLMISRTPEVTCVVV
DVSHEDPEVKFNWYVDGVEVHNAKTKPREEQYNSTYRVVSVLTVL
HQDWLNGKEYKCKVSNKALPAPIEKTISKAKGQPREPQVYTLPPS
REEMTKNQVSLTCLVKGFYPSDIAVEWESNGQPENNYKTTPPVLD
SDGSFFLYSKLTVDKSRWQQGNVFSCSVMHEALHNHYTQKSLSLS
PGK.

In some aspects the light chain of the anti-VEGF antibody comprises the sequence:

(SEQ ID NO: 2)
DIQMTQSPSSLSASVGDRVTITCSASQDISNYLNWYQQKPGKAPK
VLIYFTSSLHSGVPSRFSGSGSGTDFTLTISSLQPEDFATYYCQQ
YSTVPWTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC.

In some aspects, the anti-VEGF antibody is administered intratumorally or intraperitoneally.

In some aspects, the anti-VEGF antibody is administered intravenously.

In some aspects, the nucleic acid vector formulated with the lipopolymer is administered prior to, concurrently with, or after the anti-VEGF antibody.

In some aspects, the nucleic acid vector formulated with the lipopolymer is administered prior to, concurrently with, or after the anticancer agent.

In some aspects, an anticancer agent is administered (e.g., first), followed by the administration of the nucleic acid vector formulated with the lipopolymer (e.g., second), and followed by administration of the anti-VEGF antibody (e.g., third).

In some aspects, an anticancer agent is administered (e.g., first), followed by the administration of the nucleic acid vector formulated with the lipopolymer (e.g., second), followed by the anti-VEGF antibody (e.g. third), and followed by a surgery to remove all or part of a tissue or tumor (e.g., interval cytoreductive surgery) (e.g., fourth).

In some aspects, an anticancer agent is administered, followed by the administration of a DNA plasmid, followed by the anti-VEGF antibody, followed by an interval cytoreductive surgery.

In some aspects, the anti-VEGF antibody is administered prior to the interval cytoreductive surgery, e.g., at least about 22 days after the first administration of the anticancer agent, e.g., every week for at least about 12 weeks to up about 18 weeks.

In some aspects, the anti-VEGF antibody is administered prior to the interval cytoreductive surgery, at least about 22 days after the first administration of the anticancer agent, every week for at least about 12 weeks to up about 18 weeks.

In some aspects, the anti-VEGF antibody is administered at least about 28 days after the interval cytoreductive surgery, and at least about 22 days after the first administration of the anticancer agent, every week for at least about 9 weeks.

In some aspects, anti-VEGF antibody is administered at a dose of about 10-20 mg/kg IV (e.g., about 15 mg/kg IV). In some aspects, anti-VEGF antibody is administered at a dose of about 15 mg/kg IV.

In some aspects, the interval cytoreductive surgery (ICS) is administered at least about 28 days following the administration of an anticancer agent. In some aspects, the interval cytoreductive surgery (ICS) is administered about 28 days following the administration of an anticancer agent.

In some aspects, the interval cytoreductive surgery (ICS) is administered at least about 7 days following the administration of the DNA plasmid. In some aspects, the interval cytoreductive surgery (ICS) is administered about 7 days following the administration of the DNA plasmid.

In some aspects, interval cytoreductive surgery (ICS) is administered at least about 7 days following the administration of the DNA plasmid. In some aspects, interval cytoreductive surgery (ICS) is administered about 7 days following the administration of the DNA plasmid.

In some aspects, the interval cytoreductive surgery (ICS) is administered at least about 28 days before the administration of the anti-VEGF antibody. In some aspects, the interval cytoreductive surgery (ICS) is administered about 28 days before the administration of the anti-VEGF antibody.

In some aspects, the interval cytoreductive surgery (ICS) is administered at least about 28 days following the administration of the anti-VEGF antibody. In some aspects, the interval cytoreductive surgery (ICS) is administered about 28 days following the administration of the anti-VEGF antibody.

### IV. Anticancer Agent

In one aspect of the foregoing therapy the anticancer agent is a chemotherapeutic drug selected from the group consisting of taxanes, platinums, adriamycins, cylcophosphamide, topotecan, carmustine (BCNU) or a combination thereof. In some aspects, the anti-cancer therapy is selected from the group consisting of paclitaxel, carboplatin, docetaxel, nab-paclitaxel, doxorubicin and any combination thereof.

In some aspects, the anticancer agent is doxorubicin.

In some aspects, the anti-cancer agent comprises paclitaxel.

In some aspects, the anti-cancer agent comprises carboplatin.

In some aspects, the anti-cancer agent comprises docetaxel.

In some aspects, the anti-cancer agent comprises nab-paclitaxel.

In some aspects, the anti-cancer agent comprises.

In some aspects, the anticancer agent is administered prior to the interval cytoreductive surgery every three weeks for about 12 weeks to about 18 weeks.

In some aspects, the anticancer agent is administered prior to the interval cytoreductive surgery every three weeks for about 12 weeks to about 18 weeks.

In some aspects, the anticancer agent is administered at least about 28 days after the interval cytoreductive surgery, e.g., every 1-2 weeks (e.g., every three weeks) for about 8-10 weeks (e.g., 9 weeks).

In some aspects, the anticancer agent is administered at least about 28 days after the interval cytoreductive surgery, every three weeks for about 9 weeks.

In some aspects, the anticancer agent is selected from the group consisting of paclitaxel, carboplatin, docetaxel, nab-paclitaxel, and any combination thereof.

In some aspects, the nucleic acid vector formulated with the lipopolymer is administered prior to, concurrently with, or after the anticancer agent.

In some aspects, an anticancer agent is administered (e.g., first), followed by the administration of the nucleic acid vector formulated with the lipopolymer (e.g., second), and followed by administration of the anti-VEGF antibody (e.g., third).

In some aspects, an anticancer agent is administered (e.g., first), followed by the administration of the nucleic acid vector formulated with the lipopolymer (e.g., second), followed by the anti-VEGF antibody (e.g. third), and followed by a surgery to remove all or part of a tissue or tumor (e.g., interval cytoreductive surgery) (e.g., fourth).

In some aspects, an anticancer agent is administered, followed by the administration of a DNA plasmid, followed by the anti-VEGF antibody, followed by an interval cytoreductive surgery.

In some aspects, the anticancer agent is administered prior to the interval cytoreductive surgery. In some aspects, the anticancer agent is administered prior to the interval cytoreductive surgery every three weeks for about 12 weeks to about 18 weeks.

In some aspects, the anticancer agent is administered at least about 28 days after the interval cytoreductive surgery (e.g., every three weeks for about 9 weeks).

In some aspects, the administration of the anticancer agent comprises administering paclitaxel at a dose of about 100-200 mg/m² (about 175 mg/m²), optionally, followed by administering carboplatin at a dose of about AUC 5-6 IV.

In some aspects, the administration of the anticancer agent comprises administering docetaxel at a dose of 50-100 mg/m² (e.g., about 75 mg/m²), optionally, followed by administering carboplatin at a dose of about AUC 5-6 IV.

In some aspects, the administration of the anticancer agent comprises administering nab-paclitaxel at a dose of 200-300 mg/m² (e.g., about 260 mg/m²), optionally followed by administering carboplatin at a dose of about AUC 5-6 IV.

In some aspects, the administration of the nanoparticle prior to the interval cytoreductive surgery begins 15 days after the first administration of the anticancer agent, every week for at least about 12 weeks to about 18 weeks.

In some aspects, the administration of the anticancer agent comprises administering paclitaxel at a dose of about 150 mg/m² to about 200 mg/m² (e.g., about 175 mg/m²) followed by administering carboplatin at a dose of about AUC 4-8 IV (e.g., about AUC 5-6 IV).

In some aspects, the administration of the anticancer agent comprises administering paclitaxel at a dose of about 175 mg/m², followed by administering carboplatin at a dose of about AUC 5-6 IV.

In some aspects, the administration of the anticancer agent comprises administering docetaxel at a dose of about 50 mg/m² to about 100 mg/m² (e.g., about 75 mg/m²), followed by administering carboplatin at a dose of about AUC 4-8 IV (e.g., about AUC 5-6 IV).

In some aspects, the administration of the anticancer agent comprises administering docetaxel at a dose of about 75 mg/m², followed by administering carboplatin at a dose of about AUC 5-6 IV.

In some aspects, the administration of the anticancer agent comprises administering nab-paclitaxel at a dose of about 240 mg/m² to about 300 mg/m² (e.g., about 260 mg/m²), followed by administering carboplatin at a dose of about AUC 4-8 IV (e.g., about AUC 5-6 IV).

In some aspects, the administration of the anticancer agent comprises administering nab-paclitaxel at a dose of about 260 mg/m², followed by administering carboplatin at a dose of about AUC 5-6 IV.

### V. Methods of Treatment

Certain aspects of the disclosure are related to a method of treating a subject suffering from cancer comprising administering to the subject a combination of (i) a nanoparticle, (ii) an antibody with binding specificity for vascular endothelial growth factor (VEGF).

This present invention also provides a method for treatment of mammalian cancer or hyperproliferative disorders by intratumoral, intraperitoneal, intravenously, intravesicularly, intratracheal, intracranial or systemic administration of pharmaceutical compositions comprising a plasmid-based gene expression system and a gene delivery polymer, without a chemotherapeutic drug. The mammalian cancer is selected from a group consisting of primary or metastasized tumors of the ovary. Preferably, the nucleic acid is a plasmid-based gene expression system containing a DNA sequence which encodes interleukin-12.

The treatment of tumors with the said pharmaceutical composition (nucleic acid plus gene delivery polymer and one or more chemotherapeutic agents) results in tumor shrinkage and extension of life span. The combination of gene therapy (nucleic acid and gene delivery polymers) with chemotherapy (chemotherapeutic agents) according to the method of the present disclosure produce additive and/or synergistic efficacy. The efficacy of the method of this invention is defined as but not limited to shrinkage in tumor size or reduction in tumor density, an increase in lymphocyte count or increase in neutrophil count or improvement in survival, or all of the above. In addition, the combination of gene therapy (nucleic acid and gene delivery polymers) with chemotherapy (chemotherapeutic agents) according to the method of the present invention lowers the toxicity of the chemotherapeutic agent and reverses tumor resistance to chemotherapy. The toxicity herein is defined as any treatment related adverse effects on clinical observation including but not limited to abnormal hematology or serum chemistry or organ toxicity. Furthermore, the combination of gene therapy (nucleic acid and gene delivery polymers) with a suboptimal dose of chemotherapy (chemotherapeutic agents) according to the method of the present invention enhances the anticancer effect to a level equal to or higher than that of achieved with the optimal dose of the chemotherapeutic agent but with lesser toxicity.

New cancer treatment strategies are focused on delivering macromolecules carrying genetic information, rather than a therapeutic protein itself, allowing for the exogenously delivered genes to be expressed in the tumor environment. Methods that utilize non-viral gene delivery systems are considered safer compared to viral delivery systems, but the practical application of current polymeric systems has not been satisfactory due to poor efficiency. A strategy has recently been disclosed whereby the gene transfection efficiency of a low molecular weight PEI was enhanced by covalent attachment of cholesterol forming a water soluble lipopolymer (WSLP). See, Mol. Ther., 2001, 4, 130. IL-12 gene transfer to solid tumors with WSLP was significantly better than by the unmodified PEI and led to more significant tumor inhibition.

It is widely recognized that a single treatment strategy against cancer is generally ineffective due to the multi-factorial nature of this disease. The benefit of combination of more than one drug to maximize anticancer response is being increasingly recognized. In the present disclosure we have combined a chemotherapeutic agent with gene delivery of an anticancer gene administered locally to the tumor site to improve treatment safety and efficacy. Combining safe and efficient local delivery of an anticancer gene with a standard chemotherapeutic agent will enhance anticancer response and patient survival without augmenting toxicity. This combination therapy will reduce the chemotherapy dose and increase tumor sensitivity to the chemotherapy. In this disclosure, it is demonstrated that pharmaceutical compositions comprising an anticancer gene complexed with a gene delivery polymer, and at least one adjunctive chemotherapeutic drug is more effective than gene therapy or chemotherapy treatment administered alone. Furthermore, the combination therapy is effective against a wide variety of tumors when given by different routes of administration and does not augment toxicity over individual therapies.

Certain aspects of the disclosure are related to a combination therapy comprising: a (i) a nucleic acid vector (e.g., a plasmid) comprising a polynucleotide that encodes an interleukin-12 (IL-12) formulated with a lipopolymer (e.g., a nanoparticle); and (ii) an antibody or antigen-binding fragment thereof that specifically binds a vascular endothelial growth factor (VEGF) (anti-VEGF antibody).

Certain aspects of the disclosure are related to a method of treating a subject suffering from cancer comprising administering to the subject a combination therapy comprising: (i) a nucleic acid vector (e.g., a plasmid) comprising a polynucleotide that encodes an interleukin-12 (IL-12) formulated with a lipopolymer (e.g., a nanoparticle); and (ii) an antibody or antigen-binding fragment thereof that specifically binds a vascular endothelial growth factor (VEGF) (anti-VEGF antibody).

In some aspects, the polynucleotide encodes human IL-12.

In some aspects, nucleic acid vector (e.g., a plasmid) comprises a promoter operably linked to a nucleic acid encoding a p35 subunit of IL-12 and a promoter operably linked to a nucleic acid encoding a p40 subunit of IL 12.

In some aspects, nucleic acid vector (e.g., a plasmid) comprises an intron, a 3′UTR (e.g., hGH 3′UTR), an antibiotic resistance gene, or any combination thereof (e.g., the elements of FIG. 4).

In some aspects, the lipopolymer comprises of polyethyleneimine (PEI) covalently linked independently to cholesterol and polyethylene glycol (PEG) groups (e.g., the lipopolymer of FIG. 5).

In some aspects, the combination further comprises an anticancer agent.

In some aspects, the anticancer agent is a chemotherapeutic agent.

In some aspects, the chemotherapeutic agent is selected from the group consisting of doxorubicin, paclitaxel, carboplatin, docetaxel, nab-paclitaxel, olaparib, and any combination thereof.

In some aspects, the anticancer agent is doxorubicin.

In some aspects, the anticancer agent is paclitaxel.

In some aspects, the anticancer agent is carboplatin.

In some aspects, the anticancer agent is docetaxel.

In some aspects, the anticancer agent is nab-paclitaxel.

In some aspects, the anticancer agent is olaparib.

In some aspects, the anti-VEGF antibody is selected from the group consisting of Bevacizumab (e,g., Avastin or a biosimilar thereof) or ranibizumab (e.g., Lucentis or a biosimilar thereof).

In some aspects, the anti-VEGF antibody comprises a variable heavy chain (VH) comprising an amino acid sequence with at least about 85% identity to SEQ ID NO: 1 (e.g., 90, 95, 96, 97, 98, 99, or 100% identity to SEQ ID NO: 1) and a variable light chain (VL) comprising an amino acid sequence with at least about 85% identity to SEQ ID NO: 2 (e.g., 90, 95, 96, 97, 98, 99, or 100% identity to SEQ ID NO: 2).

In some aspect, the method further comprises a surgery to remove all or part of a tissue or tumor (e.g., interval cytoreductive surgery) in the subject.

In some aspects, the nucleic acid vector formulated with the lipopolymer is administered intratumorally or intraperitoneally.

In some aspects, the nucleic acid vector formulated with the lipopolymer is administered intravenously.

In some aspects, the anti-VEGF antibody is administered intratumorally intraperitoneally, intravesicularly, or any combination thereof.

In some aspects, the anti-VEGF antibody is administered intratumorally or intraperitoneally.

In some aspects, the anti-VEGF antibody is administered intravenously.

In some aspects, the nucleic acid vector formulated with the lipopolymer is administered prior to, concurrently with, or after the anti-VEGF antibody.

In some aspects, the nucleic acid vector formulated with the lipopolymer is administered prior to, concurrently with, or after the anticancer agent.

In some aspects, an anticancer agent is administered (e.g., first), followed by the administration of the nucleic acid vector formulated with the lipopolymer (e.g., second), and followed by administration of the anti-VEGF antibody (e.g., third).

In some aspects, an anticancer agent is administered (e.g., first), followed by the administration of the nucleic acid vector formulated with the lipopolymer (e.g., second), followed by the anti-VEGF antibody (e.g. third), and followed by a surgery to remove all or part of a tissue or tumor (e.g., interval cytoreductive surgery) (e.g., fourth).

In some aspects, an anticancer agent is administered, followed by the administration of a DNA plasmid, followed by the anti-VEGF antibody, followed by an interval cytoreductive surgery.

In some aspects, the anticancer agent is administered prior to the interval cytoreductive surgery every three weeks for about 12 weeks to about 18 weeks.

In some aspects, the anticancer agent is administered at least about 28 days after the interval cytoreductive surgery (e.g., every three weeks for about 9 weeks).

In some aspects, the administration of the anticancer agent comprises administering paclitaxel at a dose of about 100-200 mg/m² (about 175 mg/m²), optionally, followed by administering carboplatin at a dose of about AUC 5-6 IV.

In some aspects, the administration of the anticancer agent comprises administering docetaxel at a dose of 50-100 mg/m² (e.g., about 75 mg/m²), optionally, followed by administering carboplatin at a dose of about AUC 5-6 IV.

In some aspects, the administration of the anticancer agent comprises administering nab-paclitaxel at a dose of 200-300 mg/m² (e.g., about 260 mg/m²), optionally followed by administering carboplatin at a dose of about AUC 5-6 IV.

In some aspects, the administration of the anticancer agent comprises administering olaparib at a dose of about 250 mg to about 350 mg (e.g., about 300 mg) PO twice.

In some aspects, the administration of the nanoparticle prior to the interval cytoreductive surgery begins 15 days after the first administration of the anticancer agent, every week for at least about 12 weeks to about 18 weeks.

In some aspects, the nanoparticle is administered at least about 28 days following the interval cytoreductive surgery, and administration begins 15 days after the first administration of the anticancer agent, every week for at least about 9 weeks.

In some aspects, the interleukin-12 (IL-12) formulated with a lipopolymer (e.g., a nanoparticle) is administered at a dose of about 35 mg/m² to about 80 mg/m². In some aspects, the interleukin-12 (IL-12) formulated with a lipopolymer (e.g., a nanoparticle) is administered at a dose of about 50 mg/m² to about 100 mg/m². In some aspects, the interleukin-12 (IL-12) formulated with a lipopolymer (e.g., a nanoparticle) is administered at a dose of about 80 mg/m².

In some aspects, the anti-VEGF antibody is administered prior to the interval cytoreductive surgery, at least about 22 days after the first administration of the anticancer agent, every week for at least about 12 weeks to up about 18 weeks.

In some aspects, the anti-VEGF antibody is administered at least about 28 days after the interval cytoreductive surgery, and at least about 22 days after the first administration of the anticancer agent, every week for at least about 9 weeks.

In some aspects, anti-VEGF antibody is administered at a dose of about 10-20 mg/kg IV (e.g., about 15 mg/kg IV). In some aspects, anti-VEGF antibody is administered at a dose of about 15 mg/kg IV.

In some aspects, the interval cytoreductive surgery (ICS) is administered at least about 28 days following the administration of an anticancer agent.

In some aspects, the interval cytoreductive surgery (ICS) is administered at least about 7 days following the administration of the DNA plasmid.

In some aspects, interval cytoreductive surgery (ICS) is administered at least about 7 days following the administration of the DNA plasmid.

In some aspects, the interval cytoreductive surgery (ICS) is administered at least about 28 days before the administration of the anti-VEGF antibody.

In some aspects, the interval cytoreductive surgery (ICS) is administered at least about 28 days following the administration of the anti-VEGF antibody.

In some aspects, the cancer is selected from a group consisting of ovarian cancer, fallopian tube cancer, primary peritoneal cancer, breast cancer, prostate cancer, colorectal cancer, bladder cancer, brain cancer (e.g., glioblastoma), lung cancer, and any combination thereof, and metastasis of any of the cancers.

In some aspects, the cancer is selected from a group consisting of ovarian cancer, fallopian tube cancer, primary peritoneal cancer, and any combination thereof.

In some aspects, the subject is a human.

In some aspects, the subject is treated with one or more of: neoadjuvant chemotherapy (NACT), bevacizumab (BEV), GEN-1, interval cytoreductive surgery (ICS), minimal residual disease (MRD) detection by second look laparoscopy (SLL), BEV+olaparaib, and BEV+GEN-1.

In some aspects, the subject is administered (a) neoadjuvant chemotherapy (NACT), bevacizumab (BEV), and GEN-1. In some aspects, in (a) the subject is treated with NACT for 4-6 cycles. In some aspects, in (a) the subject is administered BEV on cycles 2, 3, 6, and 7. In some aspects, in (a) BEV is included with each cycle EXCEPT the following cycles: (1) Cycle 1, (2) the last cycle of neoadjuvant therapy immediately preceding ICS (maybe as C4, C4+1 or C4+2), and (3) the first cycle of adjuvant chemotherapy (i.e., first cycle after ICS). In some aspects, in (a) the subject is administered GEN-1 in weekly starting C1D15. In some aspects, each cycle is a 21 day cycle.

In some aspects, the NACT is carboplatin and paclitaxel. In some aspects, the NACT is administered once a cycle (e.g., every three weeks). In some aspects, paclitaxel is administered at a dose of about 175 mg/m² IV followed by carboplatin AUC 5-6 IV on C1D1. In some aspects, BEV is administered at a dose of about 15 mg/kg IV on day 1 of included cycles. In some aspects, GEN-1 is administered at a dose of about 80 mg/m² IP.

In some aspects, (a) is followed by (b) interval cytoreductive surgery. In some aspects, ICS will take place at least 4 weeks after the last dose of NACT from (a).

In some aspects, the subject is (c) administered neoadjuvant chemotherapy (NACT), bevacizumab (BEV), and GEN-1. In some aspects, in (c) the subject is treated with NACT for 3 cycles. In some aspects, in (c) the subject is administered BEV on cycles 2, 3, 6, and 7. In some aspects, in (c) BEV is included with each cycle EXCEPT the following cycles: (1) Cycle 1, (2) the last cycle of neoadjuvant therapy immediately preceding ICS (maybe as C4, C4+1 or C4+2), and (3) the first cycle of adjuvant chemotherapy (i.e., first cycle after ICS). In some aspects, in (c) the subject is administered GEN-1 weekly. In some aspects, each cycle is a 21 day cycle.

In some aspects, the NACT is carboplatin and paclitaxel. In some aspects, the NACT is administered once a cycle (e.g., every three weeks). In some aspects, paclitaxel is administered at a dose of about 175 mg/m² IV followed by carboplatin AUC 5-6 IV on C1D1. In some aspects, BEV is administered at a dose of about 15 mg/kg IV on day 1 of included cycles. In some aspects, GEN-1 is administered at a dose of about 80 mg/m² IP.

In some aspects, (b) is followed by (c).

In some aspects, (c) is followed by (d) minimal residual disease (MRD) detection by second look laparoscopy (SLL).

In some aspects, during the maintenance phase the subject is administered (e) BEV and olaparaib. In some aspects, during the maintenance phase if the subject is BRCA+/homologous recombination deficiency positive (HRD+) the subject is administered (e). In some aspects, in (e) BEV is administered at about 15 mg/kg IV daily for 3 weeks for a maximum of 18 cycles. In some aspects, in (e) olaparib is administered at a dose of about 300 mg PO twice. In some aspects, the (d) is followed by (e).

In some aspects, during the maintenance phase the subject is administered (f) BEV and GEN-1. In some aspects, during the maintenance phase if the subject is BRCA−/homologous recombination proficient (HRP) the subject is administered (f). In some aspects, in (f) BEV is administered at about 15 mg/kg IV every 3 weeks for a maximum of 18 cycles. In some aspects, in (f) GEN-1 is administered at a dose of about 80 mg/m² IP every 21 days for up to an additional 18 cycles. In some aspects, the (d) is followed by (f).

In some aspects, the subject is treated with one, two, three, four or more of (a), (b), (c), (d), and (e). In some aspects, the subject is treated with all of (a), (b), (c), (d), and (e).

In some aspects, the subject is treated with one, two, three, four or more of (a), (b), (c), (d), and (f).

In some aspects, the subject is treated with all of (a), (b), (c), (d), and (f).

The following examples are illustrative and do not limit the scope of the claimed aspects.

### EXAMPLES

### Example 1. GEN-1 Enhances the Activity of an Anti-VEGF Antibody in a Mouse Model

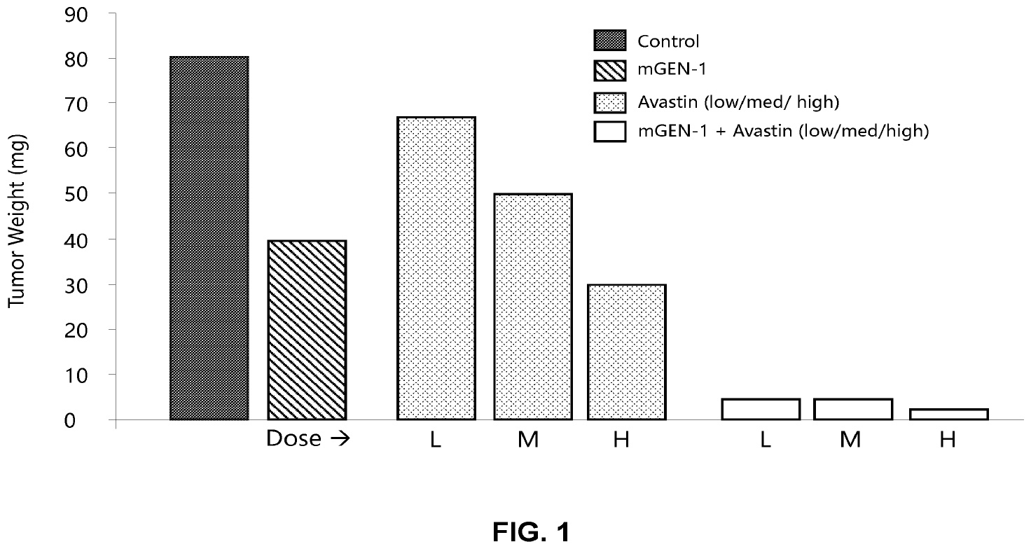

FIG. 1 shows the synergistic efficacy potential of VEGF level reduction and production inhibition by administering an anti-VEGF antibody (e.g. Bevacizumab) concurrently with GEN-1. The results were achieved by intraperitoneally injecting nude-foxn1^{nu} mice with SKOV-3 (human ovarian epithelial adenocarcinoma) cells (7×10⁶ cells). An anti-VEGF antibody was intravenously administered dosed at varying levels: 5 mg/kg (low), 10 mg/kg (medium), and 20 mg/kg (high).

The anti-VEGF antibody (e.g. Bevacizumab) was administered 9 days after initial tumor implantation and proceeded to be administered once every week for 6 weeks. mGEN-1 (100 μg DNA) was then administered intraperitoneally, beginning on the 14th day after initial tumor implantation and proceeded to be administered once every week, for 4 weeks.

After 59 days from the initial tumor implantation, the mice were euthanized and the tumors were then removed, and subsequently weighed. Efficacy improvement of a low dose anti-VEGF antibody (e.g. Bevacizumab) administration is exhibited when administered in combination with GEN-1, which improves the therapeutic index and cost. See FIG. 1.

### Example 2. GEN-1 Enhances the Activity of an Anti-VEGF Antibody in Combination with an Anticancer Agent in a Mouse Model

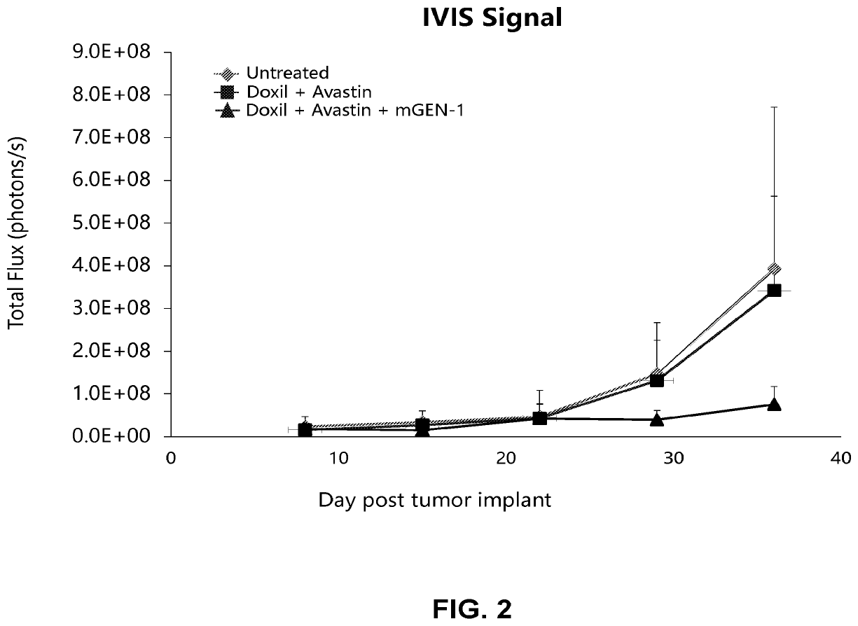

FIG. 2 shows the synergistic efficacy potential of VEGF level reduction and production inhibition by administering an anti-VEGF antibody (e.g. Bevacizumab) with an anticancer agent concurrently with GEN-1. The results were achieved by intraperitoneally injecting nude-foxn1^{nu} mice with SKOV-3-Luc (human ovarian epithelial adenocarcinoma) cells (7×10⁶ cells) in 500 μl. Doxil was administered intraperitoneally at a dosage of 7.5 mg/kg every other week, beginning 2 weeks after tumor implantation. The anti-VEGF antibody (e.g. Bevacizumab) was intravenously administered at 10 mg/kg on a weekly basis, 10 days after the tumor implantation.

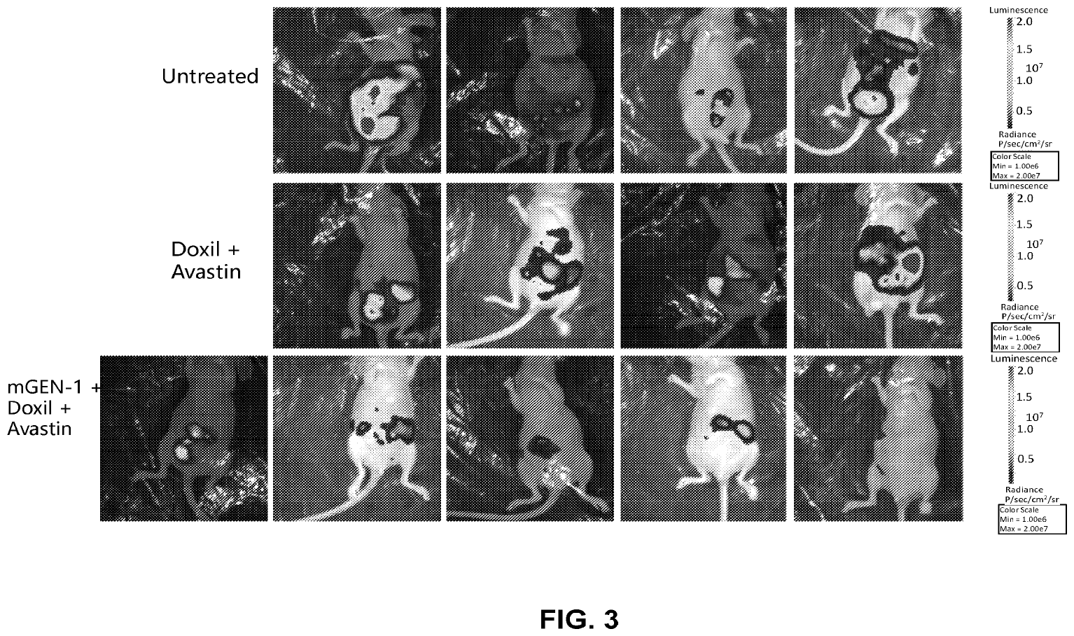

mGEN-1 was then administered intraperitoneally (100 ug DNA) on a weekly basis, beginning two weeks after the initial tumor implantation. IVIS imaging was then used to quantify tumor burden animals, as shown in FIG. 2. Whole body images of the mice via IVIS imaging are shown as FIG. 3.

### Example 3. Clinical Combination of GEN-1+NACT+Anti-VEGF Antibody

This will be a 1:1 randomized, open label, multi-center phase II trial with a safety lead in to evaluate the safety, dosing, efficacy, and biological activity of adding GEN-1 to neoadjuvant chemotherapy (NACT)+an anti-VEGF antibody (e.g. Bevacizumab) (BEV) compared to NACT+BEV alone. The NACT will be a standard regimen of carboplatin+paclitaxel administered every three weeks for 7-9 cycles. The protocol will require at least 4 cycles of neoadjuvant chemotherapy and allows up to 2 additional cycles (C4+1 and C4+2) prior to ICS at the Principal Investigator's discretion based on response and other clinical considerations. ICS will take place after a 3-4 week rest from last dose of NACT. Following at least a 4-week recovery from ICS, 3 additional adjuvant cycles of study treatments will be administered.

In addition, BEV will be included with each cycle EXCEPT the following cycles: Cycle 1, the last cycle of neoadjuvant therapy immediately preceding ICS, and the first cycle of adjuvant chemotherapy (i.e. first cycle after ICS). In the Experimental Arm, 80 mg/m² IP GEN-1 will be administered every 7 days beginning on cycle 1, day 15 (C1D15) and continue weekly though the last cycle of adjuvant therapy. At no time may BEV be given within 30 days before or after surgery. The experimental arm will add GEN-1 weekly to each cycle of NACT+BEV beginning with cycle 1 day 15. An FDA approved BEV biosimilar may be used.

A safety run-in will evaluate the safety of adding GEN-1 weekly to the NACT+BEV regimen in up to 12 subjects. This will be a standard 3+3 design with a Data Safety Monitoring Board (DSMB) evaluating cohorts of three subjects who were dosed with at least 2 cycles of NACT+BEV+GEN-1 prior to initiating the main phase of the study. The run-in patients will be randomized as well. Subjects must have received at least two cycles of chemotherapy+BEV+GEN-1 to be evaluable for safety. At least six subjects from the GEN-1 ARM must be evaluable for safety before a phase II dose of GEN-1.

Phase II of the study may begin accrual once the recommended safe dose has been determined by the DSMB from the safety phase. The study will randomize about 50 subjects. At the completion of NACT all subjects will undergo Second Look Laparoscopy (SLL) to determine if Minimal Residual Disease (MRD) positivity. SLL will be performed according to a standardized surgical approach by a gynecologic oncologist.

Maintenance treatment will be determined by BRCA+/HRD status. All subjects will receive BEV while only the BRCA+/HRD subjects will receive Olaparib in addition to BEV. Subjects on the experimental arm who are BRCA−/HRP will receive GEN-1 with BEV. All subjects will be followed for disease progression and survival.

### Study Phases

### Run-In

To ensure that the combination of NACT+BEV+GEN-1 is safe the study will enroll at least six subjects in the experimental arm before starting the main phase of the protocol. Before initiating the main phase of the study, no more than two of six of the subjects treated on the experimental arm can exhibit a dose limiting toxicity. An independent DSMB will review the safety data from subjects who were administered at least two cycles of NACT+BEV+GEN-1 and provide a recommendation regarding dose modifications, safety monitoring and dosing for the main phase of the study. A DSMB charter will specify definitions of dose limiting toxicities (DLT) for the safety phase and the responsibilities of the committee which would include dose modifications and recommending the phase II dose of GEN-1.

### Phase II

The main phase of the study may begin accrual once the recommended safe dose has been determined by the DSMB from the safety phase. The study will randomize about 50 subjects in the phase II combined (25 per arm). All subjects will be randomized to either NACT+BEV+GEN-1 or NACT+BEV alone. Subjects will receive 4-6 cycles of treatment prior to Interval cytoreductive surgery (ICS) followed by at least 2 cycles of treatment post-surgery. At the conclusion of the final cycle of chemotherapy for all subjects a SLL will take place prior to initiating maintenance phase.

### Maintenance

After completion of SLL subjects will begin maintenance treatment. This phase will commence no sooner than 4 weeks (and ideally 5-7 weeks) from the date of SLL.

All subjects will be administered BEV (or FDA approved biosimilar) every 21 days until disease progression or unacceptable toxicity or up to 15 months.

BRCA+/HRD Subject Only: Olaparib 300 mg PO twice daily will be administered to all BRCA+/HRD subjects beginning with the maintenance phase until disease progression or unacceptable toxicity or up to 24 months. In addition, BEV 15 mg/kg will be administered every 3 weeks as a single agent until disease progression or unacceptable toxicity for up to an additional 18 cycles.

BRCA−/HRP Subjects Only: GEN-1 will be administered in subjects who are BRCA−/HRP every 21 days with BEV 15 mg/kg until disease progression or unacceptable toxicity for up to an additional 18 cycles.

### Study Population

Approximately 50 (including up to 12 safety run-in) subjects with newly diagnosed, advanced ovarian cancer will be randomized.

### Inclusion Criteria

1. Subjects must have suspected diagnosis of high grade epithelial ovarian, fallopian tube, or primary peritoneal carcinoma and histologic confirmation per pre-treatment biopsies by laparoscopy, or interventional radiology or CT or ultrasound guided core biopsy. Histologic documentation of the original primary tumor is required via the pathology report.
2. Subjects must have an International Federation of Gynecology and Obstetrics (FIGO) stage of III or IV who based on standard of care clinical considerations have been determined to benefit from neoadjuvant therapy.
3. Subjects only with high grade serous adenocarcinoma histologic epithelial cell type are eligible.
4. Subjects must have adequate:
I. Bone marrow function: Absolute neutrophil count (ANC) greater than or equal to 1,500/mcl. This ANC cannot have been induced or supported by granulocyte colony stimulating factors. Platelets greater than or equal to 100,000/mcl.
II. Renal function: estimated GFR by Cockcroft-Gault >=50 ml/min. Urine dipstick showing 1+ or less proteinuria (Patients with a 2+ or greater urine dipstick reading should undergo a 24-hour urine collection and have less than 2 g protein/24 hrs.)
III. Hepatic function: Bilirubin≤1.5×ULN. SGOT (AST) and SGPT (ALT)≤3.0×ULN and alkaline phosphatase≤2.5×ULN.
IV. Neurologic function: Neuropathy (sensory and motor) less than or equal to Grade 1.
5. Subjects should be free of active infection requiring isolation, parenteral antibiotics or a serious uncontrolled medical illness or disorder within four weeks of study entry.
6. Any hormonal therapy directed at the malignant tumor must be discontinued at least one week prior to the first treatment. Continuation of hormone replacement therapy is permitted.
7. Subjects must have a performance status score of 0-1 by Eastern Cooperative Group (ECOG) criteria.
8. Subjects of childbearing potential must have a negative serum pregnancy test within 14 days prior to initiation of protocol therapy and be practicing an effective form of contraception. If applicable, subjects must discontinue breastfeeding prior to study entry.
9. Subjects must have satisfactory results for the baseline laboratory analyses and diagnostic procedures as specified in the protocol.
10. Subjects must have signed an IRB/EC approved informed consent and authorization permitting release of personal health information.
11. Subjects must be at least 18 years old.
1. Subjects who have received prior treatment with GEN-1.
2. History of allergic reactions attributed to compounds of similar chemical or biologic composition to GEN-1 or other agents used in this study.
3. Subjects who have received oral or parenteral corticosteroids within 2 weeks of study entry or who have a clinical requirement for ongoing systemic immunosuppressive therapy such as chronic steroid (prednisone equivalent of >10 mg/day) use not related to chemotherapy administration. Steroid prophylaxis for IV contrast allergy is allowed.
4. Subjects with autoimmune disease requiring immunosuppressive therapy within the last 2 years. Examples of autoimmune disease include systemic lupus erythematosus, multiple sclerosis, inflammatory bowel disease and rheumatoid arthritis.
5. Subjects with known human immunodeficiency virus (HIV) or human T-lymphotropic virus (HTLV) infections are excluded.
6. Subjects with other invasive malignancies are excluded if there is any evidence of the invasive malignancy being present within the last three years. Subjects are also excluded if their previous cancer treatment contraindicates this protocol therapy. Subjects with non-invasive malignancies such as non-melanoma skin cancer, melanoma in-situ, etc. are eligible.
7. Subjects who have received prior radiotherapy to any portion of the abdominal cavity or pelvis are excluded. Prior radiation for localized cancer of the breast, head and neck, or skin is permitted, provided that it was completed more than three years prior to registration, and the patient remains free of recurrent or metastatic disease.
8. Subjects who have received prior chemotherapy for any abdominal or pelvic tumor are excluded. Subjects may have received prior adjuvant chemotherapy for localized breast cancer, provided that it was completed more than three years prior to registration, and that the patient remains free of recurrent or metastatic disease.
9. Subjects with known active hepatitis.
10. Subjects with known nephrotic syndrome (proteinuria Grade 2 or greater).
11. Subjects with concurrent severe medical problems unrelated to the malignancy that would significantly limit full compliance with the study or expose the subject to extreme risk or decreased life expectancy.
12. Subjects with clinically significant cardiovascular disease. This includes:
   a) Uncontrolled hypertension, defined as systolic blood pressure (BP)>150 mm Hg or diastolic BP>90 mm Hg on at least two separate days. (Subjects with uncontrolled hypertension may become eligible once hypertension is under control)
   b) Myocardial infarction or unstable angina within six months prior to registration.
   c) History of serious ventricular arrhythmia (i.e., ventricular tachycardia or ventricular fibrillation) or cardiac arrhythmias requiring anti-arrhythmic medications (except for atrial fibrillation that is well controlled with anti-arrhythmic medication).
   d) QTc interval ≥450 ms on baseline ECG (electrocardiogram).
   e) Baseline ejection fraction≤50% as assessed by echocardiogram or MUGA.
   f) New York Heart Association (NYHA) Class II or higher congestive heart failure.
13. Subjects of childbearing potential, not practicing adequate contraception, subjects who are pregnant, or subjects who are breastfeeding are not eligible for this trial.
14. Subjects with history or evidence upon physical examination of CNS disease, including primary brain tumor, seizures not controlled with standard medical therapy, any brain metastases, or history of cerebrovascular accident (CVA, stroke), transient ischemic attack (TIA) or subarachnoid hemorrhage within six months of the first date of treatment on this study.
15. Subjects with a history of diverticulitis. Diverticulosis is not exclusionary.
16. Subjects having hemoptysis within the last month.
17. Subjects with any condition/anomaly that would interfere with the appropriate placement of the IP catheter for study drug administration including abdominal surgery within 4 weeks of study entry (for reasons other than IP port placement), intestinal dysfunction, fistulas or suspected extensive adhesions from prior history or finding at laparoscopy.

### Enrollment and Withdrawal Procedures

Subjects with newly diagnosed, advanced ovarian cancer will be randomized. Randomized subject will be treated with NACT+BEV+GEN-1 or NACT+BEV.

### Study Treatment and Dosing

### Allocation to Treatment

Eligible subjects will be randomized in a 1:1 ratio to either the NACT+BEV+GEN-1 or NACT+BEV alone. Since this is an open label study, the site staff, subject and Sponsor will be aware of each subject's treatment.

### Neoadjuvant Chemotherapy (NACT)

### Control Arm

NACT: Paclitaxel 175 mg/m² will be administered intravenously (IV) followed by carboplatin AUC 5-6 IV on CID1. This will be repeated every 21 days, on Day 1 for 4 cycles prior to ICS and 3 cycles after ICS. Up to an additional 2 cycles (C4+1 and C4+2) may be added at the physician's discretion prior to ICS. If there is a paclitaxel reaction, docetaxel 75 mg/m² or nab-paclitaxel (Abraxane) 260 mg/m² may be substituted per institutional guidelines. Body Surface Area (BSA) will be calculated according to local practice.

BEV will be included with each cycle EXCEPT the following cycles: Cycle 1, the last cycle of neoadjuvant therapy immediately preceding ICS, and the first cycle of adjuvant chemotherapy (i.e. first cycle after ICS). An FDA approved BEV biosimilar may be used in this study.

In the Experimental Arm GEN-1 will begin on C1D15 and continue weekly through the last cycle of adjuvant therapy.

Interval cytoreductive surgery (ICS) will take place at least 28 days after the last cycle of neoadjuvant chemotherapy and protocol therapy will resume as adjuvant chemotherapy following recovery from ICS (at least 28 days) for another 3 cycles. At no time may BEV be administered within 28-days before or after surgery.

Prophylactic dexamethasone to prevent hypersensitivity reactions will only be permitted at initial dose of NACT. BEV 15 mg/kg IV administration will be on day 1 of cycles 2, 3, 6 and 7. During the maintenance phase, BEV 15 mg/kg will be administered every 3 weeks as a single agent until disease progression or unacceptable toxicity for up to an additional 18 cycles. In total, BEV can be administered up to 24 cycles.

### Hypersensitivity Prevention

On cycle 1 day 1 all patients treated with paclitaxel should be pretreated with corticosteroids such as dexamethasone. Subsequent dosing (e.g., C2D1) corticosteroids is prohibited during chemotherapy in order to avoid blunting the effect of GEN-1. Patients who develop hypersensitivity reactions may be switched to docetaxel or abraxane.

### Dose Modification

Investigators should follow their institutional standards for dose modification due to adverse reactions from chemotherapy, BEV or olaparib. Any dose modifications, or skipped doses for GEN-1 should be approved by the study chair or study monitor. Doses of GEN-1 can be skipped for up to two weeks at any time if warranted due to chemotherapy drug-related adverse events in order to maintain the schedule. However, delays in GEN-1 dosing preferably do not delay or shift the timing of the chemotherapy dosing. In the event of Grade 3 or Grade 4 toxicities attributed to GEN-1, subsequent doses are skipped until recovery to ≤Grade 1. In general, GEN-1 can be given as long as ANC is >500 and PLT>50K otherwise dose modification may be considered.

If the subject experiences Grade 3 abdominal pain following analgesic regimen then, the dose of GEN-1 will be decreased to 60 mg/m² in all subsequent doses.

Refer to the package inserts (labeling) and local institutional guidelines for carboplatin, paclitaxel, abraxane, bevacizumab (or biosimilar), and docetaxel for complete prescribing information.

### GEN-1 (Investigational Medicinal Product)

Human IL-12 plasmid (phIL-12-005) is formulated with lipopolymer PEG-PEI-Cholesterol (PPC) in 10% lactose.

### Human IL-12 Plasmid

The phIL-12-005 plasmid contains hIL-12 gene expression cassette in a plasmid containing a Kan^{r} gene. The hIL-12 gene expression cassette of phIL-12-005 contains immediate early enhancer and promoter derived from cytomegalovirus (CMV), 5′ untranslated region (UTR), synthetic intron, p35 gene, human growth hormone (hGH) 3′ UTR and polyadenylation signal sequence, CMV promoter, 5′ UTR, synthetic intron, p40 gene, hGH 3′ UTR and polyadenylation signal sequence. The two hIL-12 subunits are individually under the control of two separate CMV promoters. (see FIG. 4)

### PEG-PEI-Cholesterol

PEG-PEI-Cholesterol (PPC) is composed of a PEI backbone to which polyethyleneglycol and cholesterol are independently attached via covalent linkages. The molecular weight of PEI, PEG and cholesterol carbonyl is 1800, 550 and 414, respectively. (see FIG. 5).

### Route of Administration & Administration of GEN-1

GEN-1 is administered after peritoneal lavage and translational specimen collection using the IP port.

25 mL of 0.9% Normal Saline will be flushed through the IP port to check for catheter patency. Heparin should not be used to flush the catheter at the time of sample collection or drug infusion.

Reconstituted GEN-1 (in the 50 mL glass vial or IV bag) is stable at room temperature for up to 24 hours. Upon confirmation of catheter patency, an IV bag containing the GEN-1 will be administered through the patient's IP catheter. GEN-1 will be infused via a catheter through gravity from the IV bag with the valve all the way open and free flowing. Typical administrations may take about 1 hour.

Following the GEN-1 infusion, a second flush at least 25 ml 0.9% Normal Saline for Injection to ensure study drug is cleared from the catheter.

To date, toxicities associated with systemic administration of recombinant IL-12 therapy have not been detected with GEN-1 administration. Phase 1 studies of GEN-1 indicate little to no systemic uptake of GEN-1 or its downstream cytokines.

Subcutaneously implantable IP silicone catheters may be used to administer GEN-1 to the peritoneal cavity. GEN-1 has been demonstrated to be compatible with silicone catheters in a prior preclinical compatibility study and in prior phase I studies. Port-A-Cath catheter (Deltec, Inc. St. Paul, MN) has been successfully used for IP delivery of GEN-1 with little to no catheter-related serious complications noted. Although a Port-A-Cath catheter is preferred, any other approved catheter with a subcutaneous port for IP delivery may also be used if suitable for aspiration of biological samples for translational studies. Bard 9.6 Fr silicone single lumen catheters for venous access or the equivalent with or without cuff from Bard Access Systems (West Amelia Earhart Drive Salt Lake City Utah) may be used. The catheter compatibility studies conducted with Port-A-Cath catheter showed that catheter exposure to GEN-1 does not significantly affect the physico-chemical properties or transfection activity of GEN-1. A similar compatibility study was performed with a Bard catheter. Subjects may be treated via pre-existing IP catheters provided they are of a similar nature to a Port-A-Cath catheter device and functional. A cathetergram may be obtained to verify intraabdominal infusion if there is concern about catheter patency or function.

Catheter Insertion: The IP catheter will be implanted per institutional standard process; the procedure and risks associated with placement of the IP catheter must be explained to the subject and an a procedure consent form will be signed by the subjects prior to placement of the catheter. The subject will undergo insertion of the IP catheter a minimum of 7 days prior to scheduled study drug administration to allow for sufficient healing and sealing around the catheter site. A semi-permanent subcutaneous access port, such as the Port-A-Cath catheter, (SIMS Deltec, Inc, St. Paul MN Inc., 55112) or equivalent device per current institutional clinical practice will be used. The port is located on the lower chest.

The study drug will be infused through this port during the course of the study. Prior to each infusion of study drug, at least 25 mL of saline will be flushed through the catheter to check for catheter patency; heparin should not be used to flush the catheter at the time of sample collection or drug infusion. Peritoneal/ascites washings for translational studies should be obtained prior to GEN-1 infusion. For those enrolled into the NACT+BEV+GEN-1 arm, the catheter may be removed at completion of GEN-1 administration at the clinician's discretion.

### Study Procedures

### Screening Overview (Day-21 to Cycle 1 Day 1)

Written informed consent must be obtained ≤21 days prior to initiation of treatment and before any procedures specifically for inclusion in the protocol are performed. All eligible subjects regardless of the arm that they are randomized to will undergo baseline evaluations prior to dosing.

Screening assessments will be performed after obtaining informed consent and within 21 days prior to the initiation of treatment. For both treatment arms, screening procedures will include a medical history, laparoscopy/biopsy, physical examination, vital signs, Eastern Cooperative Group (ECOG) performance status, ECG (electrocardiogramcluding serum pregnancy test and CA-125, and radiological imaging scans. Radiological imaging scans can be completed within 21 days prior to the initiation of treatment. Screening laboratory procedures may be repeated to assess eligibility parameters during the screening period.

Screening assessment will be collected, reviewed, and determined to be acceptable by the Principal Investigator prior to randomization and must provide sufficient time for the subject to have her IP catheter inserted. Any change in health evaluated by physical exam or vital signs should be acceptable to the Principal Investigator before study treatment is started.

Eligible subjects will be randomly assigned 1:1 to receive either NACT+BEV+GEN-1 or NACT+BEV.

### NACT and Interval Cytoreductive Surgery

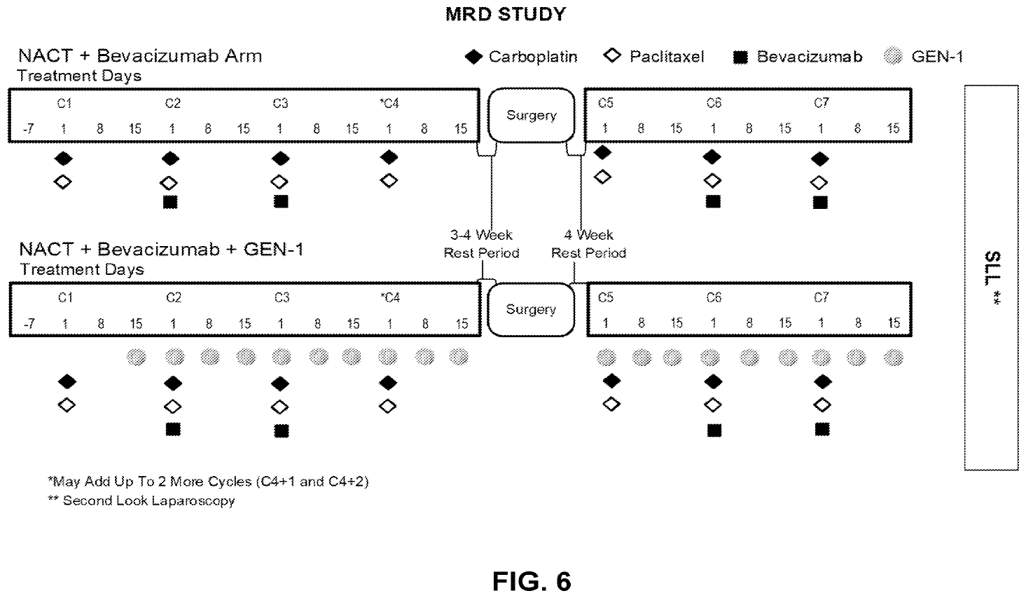

All subjects will receive 7-9 cycles standard NACT every 21 days. (See FIG. 6). BEV will be administered on C2D1, C3D1 and after ICS on C6D1, C7D1 (and additional neoadjuvant cycles if necessary, except for the cycle immediately preceding ICS). GEN-1 will be added to the regimen for subjects in the experimental arm weekly beginning C1D15. In subjects randomized to the experimental arm an IP port must be placed at least 7 days before GEN-1 dosing to allow for healing. ICS will take place 4 weeks after last dose of neoadjuvant cycle of chemotherapy (based on response per investigator's judgment). The remaining 3 cycles of the adjuvant treatment regimen will begin after at least a 4-week recovery period from ICS.

### Maintenance

All subjects will be administered BEV every 21 days until tolerance or unacceptable toxicity, for a maximum of an additional 18 cycles.

A run-in phase to ensure safety of the NACT+BEV+GEN-1 combination will evaluate at least six subjects randomized to the experimental arm in a 3+3 design. Subjects must have received at least two cycles of NACT+BEV+GEN-1 to be evaluable for safety. At least six subjects from the GEN-1 ARM must be evaluable for safety before DSMB can recommend a phase II dose of GEN-1. Generally, <2 subjects of 6 may have a dose limiting toxicity to proceed to phase II. The DSMB will review the safety data from evaluable subjects and make recommendations to the sponsor and study chair.

Subjects will be monitored for safety (with physical exams and assessment of AEs) at every treatment visit from the time of signing informed consent until at least 30 days following their last dose of chemotherapy or GEN-1. Any suspected drug related AE may be reported at any time during follow up until resolution to a grade≤2 (CTCAE v5.0).

Subjects will be monitored for antitumor activity clinically (CA-125) and by CT or MRI scan at screening, prior to ICS, and approximately 4 weeks post completion of all carboplatin+paclitaxel chemotherapy. Then subjects will be monitored clinically, via CA-125 and by CT/MRI every 3 months until progression. Investigator determined radiological progression (per RECIST v. 1.1) are observed and recorded prior to beginning alternate treatments.

SLL takes place within approximately 6-8 weeks (+1 week) after Day 1 of the last cycle of adjuvant chemotherapy. SLL is performed at least 4 weeks after last dose of BEV.

### Assessment

The presence of histopathologically confirmed MRD at SLL will be used as the primary endpoint to evaluate efficacy. For the purposes of the primary endpoint, presence of minimal residual disease (MRD) at the time of SLL will be defined as any histopathologic or cytologic evidence of viable residual carcinoma in SLL derived peritoneal biopsies or washings.

The rate of MRD at SLL is defined as the proportion of subjects who are MRD+ (microscopic only or gross disease) at the time of SLL.

For exploratory analyses residual disease at SLL is further classified as according to the following three categories: a) complete response (no evidence of disease in any biopsies or peritoneal washings); b) macroscopic MRD (surgically visible or suspicious disease confirm by histopathologic diagnosis); c) microscopic MRD (no definitive visible residual disease at SLL, but microscopically positive biopsy or positive cytology on peritoneal washings). Fisher's exact test is used to compare the difference in rate of MRD at SLL positivity (includes partial response and persistent disease) between the two groups. Eighty percent (80%) and 95% Clopper Pearson exact confidence intervals are derived for the individual treatment rates and ones with continuity correction for the difference in proportions.

Secondary endpoints will include progression free survival (PFS) and overall survival (OS). PFS is defined as the duration of time from randomization to time of investigator assessed progression or death, whichever occurs first. CT scans can also be collected. OS is defined as the time (in months) from the date of randomization to the date of death. In the absence of death confirmation or for subjects alive as of the OS cut-off date, survival time is censored at the date of last study follow-up, or the cut-off date, whichever is earlier.

This protocol allows the participation of subjects with both measurable and non-measurable disease, and as such, deviates slightly from the standard RECIST 1.1 language with respect to the usage of CA-125 in order to define biochemical response and progression. Note: “response” below refers to clinical response, not “biochemical response,” unless specifically noted as such.

What is claimed is:

1. A combination therapy comprising:
(a) a nucleic acid vector comprising a polynucleotide that encodes an interleukin-12 (IL-12) formulated with a lipopolymer; and
(b) an antibody or antigen-binding fragment thereof that specifically binds a vascular endothelial growth factor (VEGF) (anti-VEGF antibody).

2. The combination therapy of claim 1, wherein the polynucleotide encodes human IL-12.

3. The combination therapy of claim 1 or 2, wherein the nucleic acid vector comprises a promoter operably linked to a nucleic acid encoding a p35 subunit of IL-12 and a promoter operably linked to a nucleic acid encoding a p40 subunit of IL 12.

4. The combination therapy of any one of claims 1-3, wherein the nucleic acid vector comprises an intron, a 3′UTR, an antibiotic resistance gene, or any combination thereof (e.g., the elements of FIG. 4).

5. The combination therapy of any one of claims 1-4, wherein the lipopolymer comprises a polyethyleneimine (PEI) covalently linked independently to cholesterol and polyethylene glycol (PEG) groups (e.g., the lipopolymer of FIG. 5).

6. The combination therapy of any one of claims 1-5, wherein the combination further comprises an anticancer agent.

7. The combination therapy of any one of claim 6, wherein the anticancer agent is a chemotherapeutic agent.

8. The combination therapy of claim 6, wherein the anticancer agent is selected from the group consisting of doxorubicin, paclitaxel, carboplatin, docetaxel, nab-paclitaxel, olaparib, and any combination thereof.

9. The combination therapy of claim 6, wherein the anticancer agent is paclitaxel.

10. The combination therapy of claim 6, wherein the anticancer agent is carboplatin.

11. The combination therapy of claim 6, wherein the anticancer agent is docetaxel.

12. The combination therapy of claim 6, wherein the anticancer agent is nab-paclitaxel.

13. The combination therapy of claim 6, wherein the anticancer agent is olaparib.

14. The combination therapy of any one of claims 1-13, wherein the anti-VEGF antibody is selected from the group consisting of Bevacizumab or ranibizumab.

15. The combination therapy of claim 14, wherein the anti-VEGF antibody is bevacizumab.

16. The combination therapy of claim 15, wherein the anti-VEGF antibody is Avastin or a biosimilar thereof.

17. The combination therapy of claim 14, wherein the anti-VEGF antibody is ranibizumab.

18. The combination therapy of claim 17, wherein the anti-VEGF antibody is Lucentis or a biosimilar thereof.

19. The combination therapy of any one of claims 1-18, wherein the anti-VEGF antibody comprises a variable heavy chain (VH) comprising an amino acid sequence with at least about 85% identity to SEQ ID NO: 1 and a variable light chain (VL) comprising an amino acid sequence with at least about 85% identity to SEQ ID NO: 2.

20. The combination therapy of claim 19, wherein the anti-VEGF antibody comprises a variable heavy chain (VH) comprising an amino acid sequence with at least about 90%, at least about 95%, at least about 96%, at least about 97%, at least about 98%, at least about 99%, or 100% identity to SEQ ID NO: 1 and a variable light chain (VL) comprising an amino acid sequence with at least about 90%, at least about 95%, at least about 96%, at least about 97%, at least about 98%, at least about 99%, or 100% identity to SEQ ID NO: 2.

21. The combination therapy of any one of claims 1-20, wherein the nucleic acid vector comprises a plasmid.

22. The combination therapy of any one of claims 4-21, where in the 3′ UTR comprises an hGH 3′ UTR.

23. A method of treating a subject suffering from cancer comprising administering to the subject the combination therapy of any one of claims 1-22.

24. The method of claim 23, wherein the nucleic acid vector formulated with the lipopolymer is administered intratumorally or intraperitoneally.

25. The method of any one of claims 23-24, wherein the nucleic acid vector formulated with the lipopolymer is administered intravenously.

26. The method of any one of claims 23-25, wherein the anti-VEGF antibody is administered intratumorally, intraperitoneally, intravenously, intravesicularly, or any combination thereof.

27. The method of any one of claims 23-26, wherein the anti-VEGF antibody is administered intravenously.

28. The method of any one of claims 23-27, wherein the nucleic acid vector formulated with the lipopolymer is administered prior to, concurrently with, or after the anti-VEGF antibody.

29. The method of any one of claims 23-28, wherein the nucleic acid vector formulated with the lipopolymer is administered prior to, concurrently with, or after the anticancer agent.

30. The method of any one of claims 23-29, wherein an anticancer agent is administered, followed by the administration of the nucleic acid vector formulated with the lipopolymer, and followed by administration of the anti-VEGF antibody.

31. The method of any one of claims 23-30, wherein an anticancer agent is administered, followed by the administration of the nucleic acid vector formulated with the lipopolymer, followed by the anti-VEGF antibody, and followed by a surgery to remove all or part of a tissue or tumor.

32. The method of claim 31, wherein the surgery is interval cytoreductive surgery.

33. The method of any one of claims 23-32, wherein an anticancer agent is administered, followed by the administration of a DNA plasmid, followed by the anti-VEGF antibody, followed by an interval cytoreductive surgery.

34. The method of any one of claims 23-33, wherein the anticancer agent is administered prior to the interval cytoreductive surgery every three weeks for about 12 weeks to about 18 weeks.

35. The method of any one of claims 23-34, wherein the anticancer agent is administered at least about 28 days after the interval cytoreductive surgery.

36. The method of any one of claims 23-34, wherein the anticancer agent is administered every three weeks for about 9 weeks after the interval cytoreductive surgery.

37. The method of any one of claims 23-35, wherein the administration of the anticancer agent comprises administering paclitaxel at a dose of about 100-200 mg/m², optionally, followed by administering carboplatin at a dose of about AUC 5-6 IV.

38. The method of claim 37, wherein administration of the anticancer agent comprises administering paclitaxel at a dose of about 175 mg/m², optionally, followed by administering carboplatin at a dose of about AUC 5-6 IV.

39. The method of any one of claims 23-38, wherein the administration of the anticancer agent comprises administering docetaxel at a dose of 50-100 mg/m², optionally, followed by administering carboplatin at a dose of about AUC 5-6 IV.

40. The method of claim 39, wherein the administration of the anticancer agent comprises administering docetaxel at a dose of about 75 mg/m², optionally, followed by administering carboplatin at a dose of about AUC 5-6 IV.

41. The method of any one of claims 23-40, wherein the administration of the anticancer agent comprises administering nab-paclitaxel at a dose of 200-300 mg/m², optionally followed by administering carboplatin at a dose of about AUC 5-6 IV.

42. The method of claim 41, wherein administration of the anticancer agent comprises administering nab-paclitaxel at a dose of about 260 mg/m², optionally, followed by administering carboplatin at a dose of about AUC 5-6 IV.

43. The method of any one of claims 23-42, wherein the administration of the nanoparticle prior to the interval cytoreductive surgery begins 15 days after the first administration of the anticancer agent, every week for at least about 12 weeks to about 18 weeks.

44. The method of any one of claims 23-43, wherein the nanoparticle is administered at least about 28 days following the interval cytoreductive surgery, and administration begins 15 days after the first administration of the anticancer agent, every week for at least about 9 weeks.

45. The method of any one of claims 23-44, wherein the interleukin-12 (IL-12) plasmid formulated with a lipopolymer is administered at a dose of about 35 mg/m² to about 80 mg/m².

46. The method of any one of claims 23-44, wherein the interleukin-12 (IL-12) plasmid formulated with a lipopolymer is administered at a dose of about 50 mg/m² to about 100 mg/m².

47. The method of any one of claims 23-44, wherein the interleukin-12 (IL-12) plasmid formulated with a lipopolymer is administered at a dose of about 80 mg/m².

48. The method of any one of claims 1-47, wherein the lipopolymer is a nanoparticle.

49. The method of any one of claims 23-48, wherein the anti-VEGF antibody is administered prior to the interval cytoreductive surgery, at least about 22 days after the first administration of the anticancer agent, every week for at least about 12 weeks to up about 18 weeks.

50. The method of any one of claims 23-49, wherein the anti-VEGF antibody is administered at least about 28 days after the interval cytoreductive surgery, and at least about 22 days after the first administration of the anticancer agent, every week for at least about 9 weeks.

51. The method of any one of claims 23-50, wherein the anti-VEGF antibody is administered at a dose of about 10-20 mg/kg IV.

52. The method of claim 51, wherein the anti-VEGF antibody is administered at a dose of about 15 mg/kg IV.

53. The method of any one of claims 23-52, wherein the interval cytoreductive surgery (ICS) is administered at least about 28 days following the administration of an anticancer agent.

54. The method of any one of claims 23-53, wherein the interval cytoreductive surgery (ICS) is administered at least about 7 days following the administration of the DNA plasmid.

55. The method of any one of claims 23-54, wherein interval cytoreductive surgery (ICS) is administered at least about 7 days following the administration of the DNA plasmid.

56. The method of any one of claims 23-55, wherein the interval cytoreductive surgery (ICS) is administered at least about 28 days before the administration of the anti-VEGF antibody.

57. The method of any one of claims 23-56, wherein the interval cytoreductive surgery (ICS) is administered at least about 28 days following the administration of the anti-VEGF antibody.

58. The method of any one of claims 23-57, wherein the cancer is selected from a group consisting of ovarian cancer, fallopian tube cancer, primary peritoneal cancer, breast cancer, prostate cancer, colorectal cancer, bladder cancer, brain cancer, lung cancer, and any combination thereof, and metastasis of any of the cancers.

59. The method of claim 58, wherein the brain cancer is a glioblastoma.

60. The method of any one of claims 23-59, wherein the cancer is selected from a group consisting of ovarian cancer, fallopian tube cancer, primary peritoneal cancer, and any combination thereof.

61. The method of any one of claims 23-60, wherein the subject is a human.
